SEQUENCE LISTING (5 sequences)

SEQ ID NO: 1 - DNA, 744 bp, Cupriavidus necator
atgactcaaagaatagcttatgtgactggaggaatgggtggaataggaactgcaatatgccaaaggttagctaaagatggatttagagtagttgctggatgtggtccaaactctccaagaagagaaagatggcttgaacagcaaaaagctttaggttttgactttgtagcaagtgaaggaaacgttgctgactgggattcaactaagacagcttttgataaagttaaagcagaagttggagaggtagatgtattaataaataatgctggaataactagagacgttgtttttagaaaaatgacaagagcagactgggatgcagtaattgatactaatttaactagcttatttaatgtgactaagcaagtaatagacggtatggctgatagaggatggggaagaatagtaaatatatcaagtgtaaacggtcagaaaggtcaatttggacagacaaattattcaacagcaaaagctggattacatggttttactatggcattagcacaggaagttgcaacaaagggagttactgttaatactgtttctccaggatatattgcaacagatatggttaaagcaattagacaagacgttttagacaaaattgtaggaacaatacctgttaaaagacttggagagcctgaggagattgcaagtatttgcgcttggctttcatctgatgaaagtggtttttctacaggtgcagattttagtttaaatggtggtcttcacatgggttagtaa

SEQ ID NO: 2 - DNA, 26 bp, Artificial Sequence
  Forward primer
cctcctttctatggagaaatctagca

SEQ ID NO: 3 - DNA, 26 bp, Artificial Sequence
  Reverse primer
tgtagcttgacctttttaagttttga

SEQ ID NO: 4 - DNA, 22 bp, Artificial Sequence
  Forward primer
gtgtagtagcctgtgaaataag

SEQ ID NO: 5 - DNA, 38 bp, Artificial Sequence
  Reverse primer
gaggcacatttattttagctagcttactaacccatgtg

(19) United States
(12) Patent Application Publication
(10) Pub. No.: US 2021/0023030 A1
(43) Pub. Date: Jan. 28, 2021

(54) PHARMACEUTICAL COMPOSITIONS

(71) Applicant: CHAIN Biotechnology Limited, Oxford (GB)

(72) Inventors: Benjamin BRADLEY, Oxford (GB); Edward GREEN, Oxford (GB); Daniela HEEG, Oxford (GB)

(21) Appl. No.: 16/982,149

(22) PCT Filed: Mar. 21, 2019
(86) PCT No.: PCT/GB2019/050801

§ 371 Date: Sep. 18, 2020

(30) Foreign Application Priority Data
Mar. 21, 2018 (GB) 1804548.4

(51) Int. Cl. A61K 31/19; A61K 35/742; C12N 15/52; A61P 1/00

(52) U.S. Cl. CPC A61K 31/19; A61K 35/742; A61K 9/0053; A61P 1/00; C12N 15/52

(57) ABSTRACT

The invention relates to pharmaceutical compositions including probiotic compositions and methods for treating metabolic diseases, neurodegenerative diseases, neurological diseases, inflammatory or autoimmune diseases and cancer.

### FIELD

The invention relates to pharmaceutical compositions for delivering 3-hydroxybutyric acid (3-HB) to the lower gastrointestinal (GI) tract to treat various diseases including metabolic diseases, neurodegenerative diseases, neurological diseases, inflammatory or autoimmune diseases and cancer; in particular, the invention concerns probiotic compositions comprising anaerobic bacteria that produce 3-H B.

### BACKGROUND

Ketone bodies are small lipid derived molecules that serve as a circulating energy source for tissues in times of fasting or prolonged exercise. Ketone bodies are produced predominantly in the liver from fatty acids mobilized from adipocytes and are distributed via the circulation to metabolically active tissues, such as muscle or brain, where they are converted to acetyl-CoA.

(R)-3-HB, also known as D-β-hydroxybutyrate (β-OHB), is a ketone body naturally produced in the liver and circulated via the blood stream to extrahepatic tissues where it can act as a metabolic substrate during periods of carbohydrate restriction. (R)-3-HB also functions in various signalling pathways but is not normally found in the gut lumen of adults. Ingestion of 3-HB in the dissociated (acid) form is impractical. 3-HB is rapidly absorbed in the small intestine and enters the blood stream where it is distributed systemically.

Butyrate, one of the three most prevalent SCFAs found in humans, is not produced by human cells but is the fermentation product of bacteria living within the digestive tract. Clostridia are one of the major butyrate producers in the human gut. These bacteria convert dietary fibre into SCFAs, which are then released as waste products into the surrounding area where they perform critical roles for human health. Like (R)-3-HB they can serve as both energetic substrates for ATP production and signalling molecules, which are critical to many processes.

Butyrate and (R)-3-HB differ by a 3′-OH group present in the latter. Butyrate is produced in the anaerobic part of the gut by bacteria, whilst (R)-3-HB is produced predominantly in the liver from fatty acids. Butyrate and (R)-3-HB may have overlapping functions and both chemicals can be metabolised to acetyl-CoA within cells, which enters the TCA cycle for ATP production, aiding growth.

A number of ways to increase the blood concentration of 3-HB have been proposed for various therapeutic benefits.

During prolonged fasting, the plasma levels of (R)-3-HB are about five times those of free fatty acids and acetoacetic acid (Longo, V. D. & Mattson, M. P. (2014) Cell Metab. 19, 181-192).

One approach involves delivering exogenous 3-HB by ingestion of a ketone monoester in which 3-HB is linked to 1,3-butanediol (1,3-BDO). This ketone ester is either blended into a drink to mask the taste or ingested in its pure liquid form. Stereo-specific ketone esters have high manufacturing costs, and environmentally unfriendly processes are used to produce the bioactive enantiomers. It is difficult to maintain a steady dose of 3-HB by ingestion.

An alternative way to deliver exogenous 3-HB is to use a salt of 3-hydroxybutyric acid. Racemic mixtures are typically used and, therefore, around 50% of the material is biologically inactive as an energy source. These products are inherently high in salt, making the products unsuitable for the treatment of medical conditions and potentially harmful even when used for non-clinical reasons in healthy populations.

3-HB can also be produced from the breakdown of poly-hydroxybutyrate (PHB) which occurs naturally in certain bacteria as a means of energy storage. It has been proposed to use PHB to benefit animal and human health. In this approach, enzymes, co-administered or present in the gut, break down the polymer and, thereby, deliver 3-HB to the gut. However, controlling enzymatic degradation is difficult and the undefined and heterogeneous product obtained is unsuitable for clinical and nutritional applications.

Various health and clinical benefits have been shown by raising blood ketone levels. Specifically, benefits have been shown in animal models, cell models and human studies using 3-HB alone, using 3-HB esters and salts (exogenous ketones), by manipulating the diet (ketogenic diet and fasting) and by using alternate ligands for the HCA2 receptor (gpr109A, a G-protein-coupled receptor to which (R)-3-HB binds with an EC50 of 318 μM, as reported in Taggart (2005) J. Biol. Chem. 280, 26649-26652).

Increased systemic levels of 3-HB has been associated with therapeutic effects in various metabolic diseases. For example, Veech, R. L., et al. (2001) IUBMB Life 51, 241-247 discusses treatment of insulin resistance and type I diabetes with a ketogenic diet. The therapeutic application of 3-HB for treatment of metabolic diseases including type 2 diabetes is also discussed in Newman, J. C. & Verdin, E., (2014) Diabetes Res. Clin. Pract. 106, 173-181 and Newman, J. C. & Verdin, E. (2017) Annu. Rev. Nutr. 37, 51-76; and the treatment of diabetes-associated diseases such as diabetic retinopathy are described in Graff, E. C., et al., (2016), Metabolism 65, 102-113. The therapeutic application of 3-HB for treating obesity and obesity-associated diseases such as NAFLD/NASH is discussed in Puchalska, P. & Crawford, P. A., (2017), Cell Metab. 25, 262-284. Longo, V. D. & Mattson, M. P. (2014) Cell Metab. 19, 181-192 describe the effect of fasting on metabolic syndrome.

Increased systemic levels of 3-HB has been associated with therapeutic effects in various neurodegenerative diseases. For example, Kashiwaya, Y. et al., (2013) Neurobiol. Aging 34, 1530-1539 describes how a ketone ester diet exhibits anxiolytic and cognition-sparing properties, and lessens amyloid and tau pathologies in a mouse model of Alzheimer's disease. Longo, V. D. & Mattson, M. P. (2014) Cell Metab. 19, 181-192 describes various studies showing that fasting has been shown to have positive effects in treatment/models of Alzheimer's disease (AD), Parkinson's disease (PD), Huntington's disease (HD). Rahman, M. et al. (2014) Nat. Commun. 5, 3944 describes 3-HB protecting against neurodegenerative diseases via HCA2 activation that induces a neuroprotective repertoire of resident monocytes/macrophages that in chronic neurodegenerative disorders, such as AD, PD, amyotrophic lateral sclerosis (ALS), and multiple sclerosis.

Diseases linked to HCA2 are discussed in Graff, E. C., et al., (2016), Metabolism 65, 102-113. These include atherosclerosis, obesity, diabetic retinopathy and neurodegenerative diseases. HCA2 ligands have shown beneficial effects in a variety of neurological disease conditions including juvenile epilepsy, AD, ALS, HD, PD, multiple sclerosis, ischemic stroke and traumatic brain injury.

Youm, Y.-H. et al. (2015), Nat. Med. 21, 263-269 describes diseases linked to the NLRP3 inflammasome such as type 2 diabetes, atherosclerosis, multiple sclerosis, AD, age-related functional decline, bone loss and gout. This paper describes how 3-HB suppresses activation of the NLRP3 inflammasome.

Shimazu, T. et al. (2013), Science 339, 211-214 (2013) have shown suppression of oxidative stress by 3-HB, an endogenous Histone Deacetylase (HDAC) Inhibitor. Diseases in which HDAC inhibitors have shown efficacy include certain cancers such cutaneous T cell lymphoma and multiple myeloma, for which drugs have been approved (see also Ding, H. et al. (2017) Leukemia 31, 1593-1602).

Veech, R. L., et al. (2001) IUBMB Life 51, 241-247 discusses treatment of epilepsy with a ketogenic diet. Longo, V. D. & Mattson, M. P. (2014) Cell Metab. 19, 181-192 and Bruce-Keller, A. J. et al. (1999) Ann. Neurol. 45, 8-15 describe how food restriction reduces brain damage and improves behavioral outcome following excitotoxic and metabolic insults. Rahman, M. et al., (2014) Nat. Commun. 5, 3944 (2014) describes how β-hydroxybutyrate receptor HCA2 activates a neuroprotective subset of macrophages and the ketone body β-hydroxybutyrate (BHB) is an endogenous factor protecting against stroke and neurodegenerative diseases.

Graff, E. C. et al., (2016) Metabolism 65, 102-113 describe anti-inflammatory effects of the hydroxycarboxylic acid receptor 2. Hutton, H. L., et al., (2016), Nephrol. Carlton Vic 21, 736-744 describe the NLRP3 inflammasome in kidney disease and autoimmunity. These papers describe the effect of 3-HB in treating various inflammatory and autoimmune diseases.

Grabacka, M. et al., (2016) Int. J. Mol. Sci. 17 describes and references studies describing the positive effects of ketone bodies in various neuropathologic conditions including AD and PD, traumatic brain injuries and anti-inflammatory actions of 3-HB, (inflammatory cytokine production and inhibition of inflammasomes in immuno-competent cells). This paper also discusses ketones/ketogenic diets and their use as part of a strategy to treat various cancers and how 3-HB has been shown to exert a cytotoxic and growth inhibitory effect on various cancer cells including lymphoma, melanoma, neuroblastoma, kidney and thyroid cancer cells.

Miyarisan Pharmaceutical Co Ltd (Japan) produces a Clostridium butyricum (CBM 588 strain) probiotic for digestive health. This product uses a non-engineered Clostridium strain, which does not produce (R)-3-HB but produces other SCFAs including butyric acid. C. butyricum is found in the human gut microbiota and has a safe history of use as a probiotic for human and animal health.

There remains a need in the art for treatments for the above diseases. In particular, there remains a need in the art for therapeutics that increase the systemic levels of 3-HB, specifically (R)-3-HB, and that avoid problems associated with other approaches of increasing systemic levels of 3-HB such as using ketone esters, salts and diet modification (e.g., fasting/ketogenic diet)—Veech, R. L., et al. (2001) IUBMB Life 51, 241-247. Gastro-intestinal problems associated with administering esters and salts include high salt loads, high costs, short-lived spiked peaks and bioavailability.

### SUMMARY OF THE INVENTION

In a first aspect, the invention provides a pharmaceutical composition for use in a method of treating a metabolic, neurodegenerative, neurological, inflammatory or autoimmune disease, disorder or condition or cancer in a subject, wherein the composition comprises a 3-hydroxybutyric acid (3-HB) delivery means, 3-HB or a combination thereof and the method comprises delivering the 3-HB delivery means, the 3-HB or combination thereof to the lower gastrointestinal (GI) tract.

In a second aspect, the invention provides a method of treating a metabolic, neurodegenerative, neurological, inflammatory or autoimmune disease, disorder or condition or cancer in a subject comprising administering to the subject a pharmaceutical composition comprising a 3-hydroxybutyric acid (3-HB) delivery means, 3-HB or a combination thereof wherein the 3-HB delivery means, the 3-HB or combination thereof is delivered to the lower gastrointestinal (GI) tract.

### BRIEF DESCRIPTION OF THE FIGURES

FIG. 1A shows the native acid production metabolic pathways in Clostridium.

FIG. 1B shows the acid production metabolic pathways in Clostridium after the introduction of a non-native (R)-3-hydroxybutyryl-CoA dehydrogenase.

FIG. 2 shows the codon optimised DNA sequence for: the phaB gene from Cupriavidus necator.

FIG. 3 details the plasmid map for pfdx_phaB in pMTL83251 (C. butyricum)

FIG. 4A shows the production of (R)-3-HB, butyrate and acetate produced by wildtype C. butyricum (wt).

FIG. 4B shows the production of (R)-3-HB, butyrate and acetate produced by genetically engineered C. butyricum (CHN-1-phaB).

FIG. 5 shows the total CFU (A) and spores (heat-resistant CFU) (B) produced by wildtype C. butyricum and genetically engineered C. butyricum (CHN-1-phaB) measured as CFU/mL over time.

FIG. 6 shows the percentage of spores to vegetative cells produced by wildtype C. butyricum and genetically engineered C. butyricum (CHN-1-phaB) over time.

FIG. 7 shows total viable counts of CHN-1 on modified BIM expressed as colony forming units/mL. CFU/mL are shown as the mean of three independent experiments with error bars representing standard deviation.

FIG. 8 shows heat-resistant counts on modified BIM expressed as colony forming units/mL. CFU/mL are shown as the mean of three independent experiments with error bars representing standard deviation.

FIG. 9 shows pH within colonic simulation measured at different time points.

FIG. 10 shows the presence of acetate (mM) in colonic simulation at selected time-points.

FIG. 11 shows the presence of butyrate (mM) in colonic simulation at selected time-points.

FIG. 12 shows the production of (R)-3-HB in colonic simulation at 24 hours for combined experimental replicates (A) and for each experimental replicate (B).

FIG. 13 shows 16s-23s intergenic spacer region-specific PCR using oligonucleotide ISR-F and ISR-R to detect CHN-1 in bioreactors.

FIG. 14 shows phaB specific PCR using oligonuletide phaB-F and phaB-R to detect CHN-1 in bioreactors.

FIG. 15 shows data from an intestinal organoid model. Relative expression (mRNA) levels, versus unstimulated organoids, of inflammatory factors NF-κB (A) and TNFα (B) are provided when organoids are incubated with TNFα, TNFα and butyrate and/or (R)-3-HB.

FIG. 16 shows data from an intestinal organoid model. Relative expression (mRNA), versus unstimulated organoids, for inflammatory markers IL-10 (A), IL-23 (B), TNF-α (C), IL-1β (D), TGF-β1 (E), IL-6 (F) and NF-κβ (G) is shown when organoids are stimulated with TNF-α alone or together with (R)-3-HB.

FIG. 17 shows relative mRNA expression level of IL-23 in organoids treated with 60 ng/mL TNF-α and increasing concentrations of butyrate or (R)-3-HB.

FIG. 18 shows colony forming units per mL of spores of CHN-1 on RCM agar plates after incubation in stomach and small intestine conditions. Data points represent the mean of three independent experiments with error bars showing the standard deviation.

FIG. 19 shows total bacterial burden expressed as CFU per gram colon tissue (A) and faeces (B).

FIG. 20 shows spore burden expressed as CFU per gram colon tissue (A) and faeces (B).

FIG. 21 shows calculated vegetative cell count expressed as CFU per gram colon tissue (A) and faeces (B).

FIG. 22 shows Metabolite level (R)-3-hydroxybutyrate levels in colon and plasma expressed as relative concentration level vs control. The mean background level measured at day −1 for colon and plasma samples was set to 1 (dashed line; control) and the relative concentration of (R)-3-hydroxybutyrate was calculated (grey bars).

### DETAILED DESCRIPTION

The invention described herein is based upon the inventors' surprising discovery that delivery of 3-HB to the lower gastrointestinal (GI) tract, in particular the colon, and specifically delivery of the (R)-isomer (R)-3-hydroxybutyrate ((R)-3-HB) using genetically engineered anaerobic bacteria, results in elevated levels of 3-HB in the blood.

Ketones have been shown to be metabolised in the colon (Roediger W E, Gastroenterology, 1982 August; 83(2):424-9). Further, butyrate is rapidly oxidised in colonocytes and does not appear in blood despite high luminal concentrations (Cummings J H, Short chain fatty acids in human large intestine, portal, hepatic and venous blood, Gut. 1987 October; 28(10):1221-7). Being structurally similar to Butyrate and having similar recognised functional activities, it would be predicted that, like Butyrate, any 3-HB in colonocytes would not be transported to the blood.

Due to its hydroxyl group, 3-HB readily forms hydrogen bonds that contribute to making it more soluble in water than butyrate. It is predicted that 3-HB would be less likely to cross the apical membrane in the lumen of the colon compared to butyrate.

3-HB can also be metabolised by certain gut bacteria. For example, bacillus have transporters to take up 3-HB (Shen, Y.-C. & Shaw, G.-C., 2015, FEMS Microbiol. Lett. 362), thus reducing the amount available to translocate into the blood.

The upper GI tract has a different structure compared to the lower GI tract, containing an increased surface area owing to surface villi which make it the major site of nutritional uptake. SCFAs are not the major source of energy for small intestine epithelium. Therefore, whilst 3-HB is known to be absorbed in the upper GI tract and reach the blood when delivered in high concentrations orally, it is unexpected that 3-HB delivered to the lower GI tract, preferably the large intestine such as the colon, should reach the blood circulation because this region exhibits lower ability to absorb nutrients and greater ability to metabolise nutrients compared to the small intestine, for example.

The invention thus provides an improved pharmaceutical composition for delivering 3-HB and increasing levels of 3-HB in the blood. In particular, the invention provides an improved pharmaceutical composition for treating diseases known to be responsive to elevated concentrations of 3-HB in the blood. The pharmaceutical composition can be administered enterally, preferably orally, and is well-tolerated. The pharmaceutical composition exhibits improved patient compliance compared to adherence to a ketogenic diet or fasting, for example. The invention avoids problems with ingestion of ketone esters and ketone salts, such as high salt loads, high costs associated with the ketone esters and short-lived spiked peaks and reduced bioavailability. The invention provides more sustained delivery and improved bioavailability of 3-HB.

The inventors have also shown that 3-HB, and specifically the (R)-isomer (R)-3-hydroxybutyrate ((R)-3-HB), is a potent anti-inflammatory agent that acts on a number of different inflammatory cytokines and signalling molecules. In particular, the inventors have shown that 3-HB downregulates certain pro-inflammatory cytokines and proteins (e.g. TNF-α, IL-23, IL-6, IL-1β, IL-12 and MMP9) and upregulates certain anti-inflammatory cytokines (e.g. IL-10, TGF-β1). The cytokines and proteins involved have been implicated in a number of inflammatory diseases, disorders or conditions.

3-HB is a chiral compound having two isomers, (R)-3-HB and (S)-3-HB. 3-HB according to the invention can be an individual isomer, a racemic mixture of isomers or a non-racemic mixture of isomers. A racemic mixture of (R)-3-HB and (S)-3-HB can have about 50%/wt (R)-3-HB and about 50%/wt (S)-3-HB. Alternatively, at least about 50, 60, 70, 80 or 90%/wt of the 3-HB can be (R)-3-HB, the remainder being (S)-3-HB. Preferably, substantially all or 100%/wt of the 3-HB can be (R)-3-HB.

The molar ratio of (R)-3-HB to (S)-3-HB can be greater than 5:1, greater than 10:1, greater than 50:1, or greater than 100:1. In one embodiment the ratio of (R)-3-HB to (S)-3-HB is in the range of about 100-5:1, 100-50:1, 100-20:1, 50-5:1, 20-5:1, 15-5:1 or about 15-10:1.

3-HB is available commercially as a pure enantiomer in the (R) or (S)-form or as a racemic mixture of (R)-3-HB and (S)-3-HB. 3-HB can also be produced by methods known in the art. Preferably, 3-HB can be produced by fermentation of anaerobic bacteria genetically engineered to produce 3-HB. 3-HB can be isolated by methods known in the art. Preferably, 3-HB can be produced by fermentation of novel Clostridium strains described herein that produce chiral compounds. For example, 3-HB that can be 100%/wt (R)-3-HB can be produced by fermenting a Clostridium species, preferably Clostridium butyricum, comprising a heterologous gene capable of expressing (R)-3-hydroxybutyryl-CoA dehydrogenase. Increased titres can be achieved by the simultaneous introduction of heterologous genes capable of expressing butyrate kinase and phosphotransbutyrylase. The introduction of the heterologous gene capable of expressing (R)-3-hydroxybutyryl-CoA dehydrogenase results in the production of the (R) form of 3-hydroxybutyryl-CoA. Native reductase enzymes then convert (R)-3-hydroxybutyryl-CoA to (R)-3-HB. Alternatively, 3-HB that can be at least about 90%/wt (R)-3-HB with the remainder being (S)-3-HB can be produced by fermenting a Clostridium species, preferably, Clostridium butyricum comprising a heterologous gene capable of expressing (R)-3-hydroxybutyryl-CoA dehydrogenase. Increased 3-HB titres can be achieved via the introduction of a heterologous gene capable of expressing a propionyl-CoA transferase (PCT). The introduction of the heterologous (R)-3-hydroxybutyryl-CoA dehydrogenase and propionyl-CoA transferase gene results in the production of (R)-3-HB and (S)-3-HB ata ratio of about 10:1.

3-HB can be in the form of a pharmaceutically acceptable salt or solvate. “3-HB” as used herein refers to 3-HB or a salt thereof. A “pharmaceutically acceptable salt” as referred to herein, is any salt preparation that is appropriate for use in a pharmaceutical application. Pharmaceutically acceptable salts include amine salts, such as N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine, tris(hydroxymethyl)aminomethane and the like; alkali metal salts, such as lithium, potassium, sodium and the like; alkali earth metal salts, such as barium, calcium, magnesium and the like; transition metal salts, such as zinc, aluminum and the like; other metal salts, such as sodium hydrogen phosphate, disodium phosphate and the like; mineral acids, such as hydrochlorides, sulfates and the like; and salts of organic acids, such as acetates, lactates, malates, tartrates, citrates, ascorbates, succinates, butyrates, valerates, fumarates and the like.

A “3-HB delivery means” can mean any chemical or biological means for delivering 3-HB or a salt thereof to the lumen of the GI tract. Suitable examples include a biological delivery system that delivers the 3-HB to the lumen of the GI tract or a prodrug of 3-HB.

By “biological delivery system” is meant a biological agent, such as a microbiological agent, preferably a bacterial agent that can be administered orally and is capable of producing 3-HB. Preferably, the biological delivery system can be genetically engineered anaerobic bacteria capable of producing 3-HB. The bacteria may produce 3-HB as either the sole fermentation product or in combination with short chain fatty acids (SCFAs), such as acetate and/or butyrate.

Compositions of the invention can comprise genetically engineered anaerobic bacteria that produce 3-HB and an orally ingestible carrier. The composition can deliver 3-HB to a subject. Once orally ingested the bacteria will subsequently grow in the subject and produce and secrete 3-HB into the anaerobic parts of the gastrointestinal tract. The bacteria may secrete 3-HB as it transits through the gut or when it becomes attached to the epithelial/mucosal cell wall lining.

The bacteria can be anaerobic bacteria. Anaerobic bacteria are bacteria that can survive in an oxygen limited (hypoxic) environment or a completely oxygen depleted (anoxic) environment. These include obligate anaerobes, which are bacteria that are harmed by the presence of oxygen and can only grow in anaerobic (no oxygen) environments; aerotolerant bacteria, which can survive in an aerobic environment (with oxygen) but cannot use molecular oxygen as a terminal electron acceptor in their respiratory pathways; and facultative anaerobes, which can survive in both aerobic and anaerobic environments and can use molecular oxygen or another molecule as a terminal electron acceptor in their respiratory pathways, depending on availability of their preferred electron acceptor. Preferably, the bacteria are obligate anaerobes.

In one embodiment the bacteria are Clostridia. The introduction of a non-native gene capable of expressing (R)-3-HB dehydrogenase ((R)-3-HBD) results in a Clostridial strain that can produce (R)-3-HB. The engineered Clostridia produce (R)-3-hydroxybutyryl-CoA. Native PTB and BUK enzymes, if present, can convert (R)-3-hydroxybutyryl-CoA into (R)-3-HB. (R)-3-HB is secreted into the gut.

Clostridia that naturally produce butyrate as the main fermentation product have now been adapted to produce (R)-3-HB either instead of, or in combination with butyrate. The Clostridia may also produce other useful fermentation products such as acetate, propionate, vitamins and bacteriocins.

Bacteria that are part of the natural gut microbiota are preferred, i.e. those bacteria that are naturally found in the gut. Bacteria that naturally produce butyrate are also preferred.

Clostridia are a preferred class of bacteria for including in the compositions. Clostridia can include but are not limited to Clostridiaceae, Christensenellaceae, Eubacteriaceae, Lachnospiraceae, Peptostreptococcaceae, Ruminococcacea. Preferably the bacteria present are from cluster I, IV and/or XlVa of Clostridia. Preferably the bacteria are Clostridia frequently detected in the lower gastrointestinal tract. For example, species detected in the lower gastrointestinal tract include:

Bacteria from the genus Clostridium (cluster 1), the preferred species for including in the composition include, but are not limited to, C. acetobutylicum, C. arbusti, C. aurantibutyricum, C. beijerinckii, C. cellulovorans, C. cellulolyticum, C. thermocellum, C. thermobutyricum, C. pasteurianum, C. kluyveri, C. novyi, C. saccharobutylicum, C. thermosuccinogenes, C. thermopalmarium, C. saccharolyticum, C. saccharoperbutylacetonicum, C. tyrobutyricum, C. tetanomorphum, C. magnum, C. ljungdahlii, C. autoethanogenum, C. butyricum, C. puniceum, C. diolis, C. 5 homopropionicum and/or C. roseum;

Bacteria from the genera Christensenellaceae, Eubacteriaceae, and Lachnospiraceae (cluster XlVa), the preferred species for including in the composition include, but are not limited to, Roseburia intestinalis, Roseburia bromii, Eubacterium rectale, Eubacterium hallii, Anaerostipes spp., Butyrivibrio spp. and/or Coprococcus spp; and

Bacteria from the genus Ruminococcacea (cluster IV), the preferred species for including in the composition include, but are not limited to, Faecalibacterium prausnitzii.

Preferably the species in the composition is C. butyricum.

Preferably the Clostridia are butyrate producers. Well-known clostridial butyrate producers include Anaerostipes spp., Butyrivibrio spp., Coprococcus spp., Roseburia spp., Eubacterium rectale- and Eubacterium hallii-related species.

Preferably the Clostridium species in the composition are capable of sporulation, preferably C. butyricum. In a preferred embodiment, the strains are DSM10702 and ATCC19398.

The engineered bacteria can comprise a non-native gene capable of expressing (R)-3-HBD. Genes capable of expressing (R)-3-HBD (EC1.1.1.36) are selected from but are not restricted to genes from organisms including Ralstonia eutropha, (Cupriavidus necator), Bacillus sp, Klebsellia sp, Pseudomonas sp, for example phbB and phaB.

Suitable genes include UniProt Accession Nos. P14697 (PHBB_CUPNH), P50203 (PHAB_ACISR), A0A060V147 (A0A060V147_KLESP), C1D6J5 (C1D6J5_LARHH), F8GXX8 (F8GXX8_CUPNN), F8GP10 (F8GP10_CUPNN), G0ETI7 (G0ETI7_CUPNN), A9LLG6 (A9LLG6_9BACI), A0A0E0VPS5 (A0A0E0VPS5_STAA5), D5DZ99 (D5DZ99_BACMQ), and V6A8L4 (V6A8L4_PSEAI)

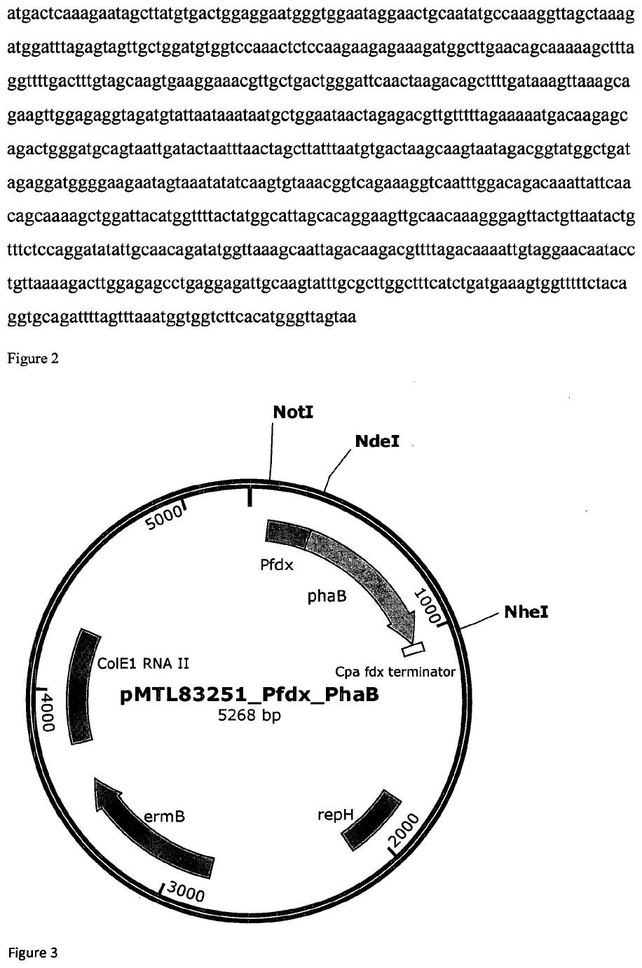

In one embodiment the (R)-3-HBD gene is phaB. The sequence of the phaB gene can be codon optimised for the specific Clostridium species used. The sequence of phaB may comprise the sequence as shown in FIG. 2 (SEQ ID NO:1).

The nucleic acid encoding the non-native (R)-3-HBD may comprise a sequence which has at least 60%, 70%, 80%, 90%, 95% or 99% sequence identity with the phaB sequence of FIG. 2 (SEQ ID NO:1).

A number of methods are available to determine identity between two sequences. A preferred computer program to determine identity between sequences includes, but is not limited to BLAST (Atschul et al, Journal of Molecular Biology, 215, 403-410, 1990). Preferably the default parameters of the computer programs are used.

In Clostridia, native enzymes can catalyse 3-hydroxybutyrate reductase reactions. Therefore, in one embodiment the Clostridium species comprise genes that encode enzymes able to convert (R)-3-hydroxybutyryl-CoA to (R)-3-HB.

Native enzymes, such as, PTB and BUK, convert (R)-3-hydroxybutyryl-CoA into (R)-3-HB via (R)-3-hydroxybutyrate-phosphate. Therefore, the genetically engineered bacteria can have native genes encoding for PTB and BUK and a non-native gene encoding (R)-3-HBD.

The Clostridium species may also comprise further non-native genes such as those encoding for PTB, BUK, PCT and/or BUT.

The Clostridium species can comprise one or more non-native genes encoding reductive enzymes able to convert (R)-3-hydroxybutyryl-CoA to (R)-3-HB, such as ptb and buk. These genes may come from organisms including but not limited to Bacillus species, E. coli, or from other species of Clostridia.

Additionally, the Clostridium species may comprise one or more native or non-native genes encoding enzymes to produce SCFA's, such as PCT or BUT. For example, a PCT from Clostridium propionicum can be engineered into a strain to catalyse the CoA transfer reaction between (R/S)-3-hydroxybutyrate-CoA and acetate.

The term “non-native gene” refers to a gene that is not in its natural environment, and includes a gene from one species of a microorganism that is introduced into another species of the same genus.

The non-native genes may be codon optimised for Clostridia and/or placed under the control of promoters that enable controllable expression of the gene in Clostridia. The expression levels of the enzymes can be optimised by controlling gene expression with inducible promoters and/or promoters with different strength. In one embodiment the non-native genes are placed under the control of a native Clostridia promoter, for example a ferredoxin or thiolase promoter. Other suitable promoters would be known to the person skilled in the art.

The non-native genes can be introduced in Clostridium strains by standard plasmid transformation techniques known in the art for producing recombinant microorganisms i.e. conjugation or electroporation. By way of example only, plasmid transformation is achieved by conjugation.

Non-native genes, including (R)-3-HBD, may be integrated into the chromosome of Clostridia using gene integration technology known to persons skilled in the art.

Clostridia are anaerobic bacteria with a fermentative metabolism that naturally convert carbohydrates into a variety of reduced fermentation products. The bacteria have unique metabolic pathways and biochemistry for producing three and four carbon (C3/C4) chemicals.

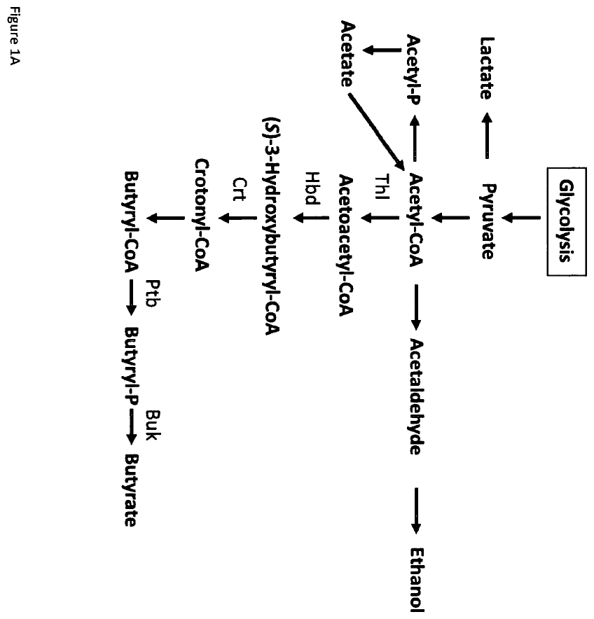

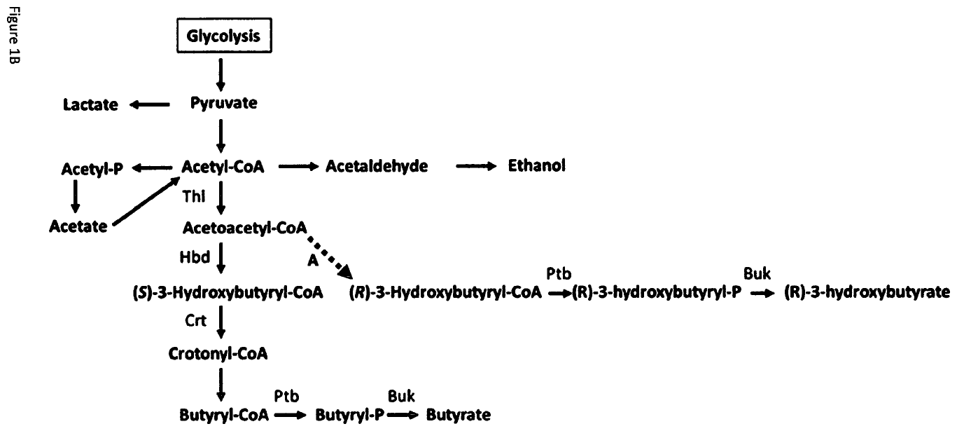

The metabolic pathway of a genetically engineered Clostridium strain is detailed in FIG. 1B. The genetically engineered Clostridium sp. carries a heterologous (R)-3-HBD (Enzyme A in FIG. 1B) that converts acetoacetyl-CoA into (R)-3-hydroxybutyryl-CoA. The (R)-specific 3-hydroxybutyryl-CoA dehydrogenase competes with the native HBD enzyme for the substrate (acetoacetyl-CoA). The native crotonase (Crt) enzyme has no or only low activity towards the (R)-form of 3-hydroxybutyryl-CoA, allowing (R)-3-hydroxybutyryl-CoA to be converted to (R)-3-HB via native enzymes, such as PTB and BUK or BUT. Enzymes PTB and BUK are specific for the R-form and convert (R)-3-hydroxybutyryl-CoA into (R)-3-HB via (R)-3-hydroxybutyryl-phosphate.

The pathway used will depend on the Clostridium species. In some species, typically those found in the Clostridiaceae family (cluster I), which includes the Clostridium genus (including C. butyricum) the final step requires two enzymes, PTB and BUK. In other species, typically those found in the Lachnospiraceae family (cluster XlVa) and Ruminococcaceae family (cluster IV) the final step requires one enzyme, BUT. Some Clostridia carry enzymes of both systems allowing them to convert (R)-3-hydroxybutyryl-CoA to (R)-3-HB.

If PTB, BUK and/or BUT are not present in the native probiotic strain, then heterologous genes encoding these enzymes can be expressed in the engineered strain.

An alternative route to produce (R)-3-HB in genetically engineered anaerobic bacteria is by the introduction of further non-native genes encoding, for example a thioesterase, i.e. TesB from E. coli. These enzymes can convert (S)- and (R)-3-hydroxybutyryl-CoA into (S)- and (R)-3-HB, respectively.

The Clostridium probiotic can be prepared by fermentation carried out under suitable conditions for growth of the bacteria. After fermentation, the bacteria can be purified using centrifugation and prepared to preserve activity. The bacteria in the composition are provided as viable organisms. The composition can comprise bacterial spores and/or vegetative cells. The bacteria can be dried to preserve the activity of the bacteria. Suitable drying methods include freeze drying, spray-drying, heat drying, and combinations thereof. The obtained powder can then be mixed with one or more pharmaceutically acceptable excipients as described herein.

The spores and/or vegetative bacteria may be formulated with the usual excipients and components for oral administration, as described herein. In particular, fatty and/or aqueous components, humectants, thickeners, preservatives, texturing agents, flavour enhancers and/or coating agents, antioxidants, preservatives and/or dyes that are customary in the pharmaceutical and food supplement industry. Suitable pharmaceutically acceptable carriers include microcrystalline cellulose, cellobiose, mannitol, glucose, sucrose, lactose, polyvinylpyrrolidone, magnesium silicate, magnesium stearate and starch, or a combination thereof. The bacteria can then be formed into a suitable orally ingestible form, as described herein. Suitable orally ingestible forms of probiotic bacteria can be prepared by methods well known in the pharmaceutical industry.

In one embodiment, the anaerobic bacteria that produce 3-HB can be present in the pharmaceutical composition in a wide range of concentrations provided the bacteria are present in an amount sufficient to provide the desired therapeutic effect. Preferably the bacteria are present in the pharmaceutical composition in an amount equivalent to between 1×10⁵ to 1×10¹¹ colony forming units/g (CFU/g) of dry composition, more preferably the bacteria are present in an amount equivalent to between 1×10⁸ to 1×10¹¹ CFU/g of dry composition, preferably 1.5×10⁸ to 1×10¹¹. When the composition is in the form of a tablet the bacteria may be present in an amount of 2×10⁵ to 6×10⁷ CFU per tablet, preferably from about 3×10⁵ to 5×10⁷ CFU per tablet or between about 1×10⁸ and about 1×10¹¹ CFU/tablet, preferably about 1×10⁹ to about 1×10¹⁰ CFU/tablet. Preferably, the bacteria grow and metabolise in the colon and deliver between about 200 μm and 20 mM 3-HB to the gut lumen, preferably between about 5 mM and 10 mM, preferably about 10 mM.

While it is possible for the 3-HB delivery means, 3-HB or a combination thereof to be administered alone, it is preferable for the 3-HB delivery means, 3-HB or a combination thereof to be present in a pharmaceutical composition. Consequently, the invention provides a pharmaceutical composition comprising a 3-HB delivery means, 3-HB ora combination thereof for use in the methods of treating a disease, disorder or condition in a subject as described herein, wherein the pharmaceutical composition is formulated to deliver the 3-HB delivery means, 3-HB or combination thereof the lower GI tract.

The present invention includes pharmaceutical compositions comprising at least one pharmaceutically acceptable carrier, and optionally other therapeutic and/or prophylactic ingredients.

The pharmaceutical compositions of the invention are administered such that a therapeutically effective amount of 3-HB is delivered and by any of the accepted modes of administration for agents that serve similar utilities.

Pharmaceutical compositions include those suitable for oral or rectal administration. Preferably, administration is oral using a convenient daily dosage regimen that can be adjusted according to the degree of affliction.

Pharmaceutical compositions of the invention can be prepared with one or more conventional adjuvants, carriers, or diluents and placed into dosage forms, such as unit dosages. The pharmaceutical compositions and dosage forms can be comprised of conventional ingredients in conventional proportions and the dosage forms can contain any suitable effective amount of the active agent (3-HB as described herein) commensurate with the intended daily dosage range to be employed.

Pharmaceutical compositions may take any of a number of different forms depending, in particular, on the manner in which it is to be used. Thus, for example, the agent or composition may be in the form of a powder, tablet, capsule, liquid, cream, gel, hydrogel, foam, micellar solution, liposome suspension or any other suitable form that may be administered to a person or animal in need of treatment. It will be appreciated that the carrier of the pharmaceutical composition according to the invention should be one which is well-tolerated by the subject to whom it is given.

A “pharmaceutically acceptable carrier” as referred to herein, is any known compound or combination of known compounds that are known to those skilled in the art to be useful in formulating pharmaceutical compositions.

In one preferred embodiment, the pharmaceutically acceptable carrier may be a solid, and the composition may be in the form of a powder or tablet. A solid pharmaceutically acceptable carrier may include one or more substances which may also act as flavouring agents, buffers, lubricants, stabilizers, solubilizers, suspending agents, wetting agents, emulsifiers, dyes, fillers, glidants, compression aids, inert binders, sweeteners, preservatives, dyes, coatings, or tablet-disintegrating agents. The carrier may also be an encapsulating material. In powders, the carrier is a finely divided solid that is in admixture with the finely divided active agents according to the invention. In tablets, the active agent may be mixed with a carrier having the necessary compression properties in suitable proportions and compacted in the shape and size desired. The powders and tablets preferably contain up to 99% of the active agents. Suitable solid carriers include, for example, calcium phosphate, magnesium stearate, talc, sugars, lactose, dextrin, starch, gelatine, cellulose, polyvinylpyrrolidine, low melting waxes and ion exchange resins. In another embodiment, the pharmaceutically acceptable carrier may be a gel and the composition may be in the form of a cream or the like.

The carrier may include one or more excipients or diluents. Examples of such excipients are gelatin, gum arabicum, lactose, microcrystalline cellulose, starch, sodium starch glycolate, calcium hydrogen phosphate, magnesium stearate, talcum, colloidal silicon dioxide and the like.

However, in another preferred embodiment, the pharmaceutically acceptable carrier may be a liquid, and the pharmaceutical composition is in the form of a solution. Liquid carriers are used in preparing solutions, suspensions, emulsions, syrups, elixirs and pressurized compositions. The active agent according to the invention may be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid carriers for oral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil).

Pharmaceutical compositions of the invention may be administered orally in the form of a sterile solution or suspension containing other solutes or suspending agents (for example, enough saline or glucose to make the solution isotonic), bile salts, acacia, gelatine, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions, including capsules containing liquid forms, all of which are known to those skilled in the art.

Pharmaceutical compositions of the invention can also be formulated for rectal administration including suppositories and enema formulations. In the case of suppositories, a low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify. Enema formulations can be semi-solid including gels or ointments or in liquid form including suspensions, aqueous solutions or foams, which are known to those skilled in the art.

Other suitable pharmaceutical carriers and their formulations are described in Remington: The Science and Practice of Pharmacy 22nd Edition, The pharmaceutical Press, London, Philadephia, 2013.

Pharmaceutical compositions of the invention can be formulated as modified-release dosage forms. By “modified release” is meant that the dosage forms are formulations where the rate and/or site of release of the active agent(s) are different from that of the immediate release dosage form administered by the same route. This modification is achieved by special formulation design and/or manufacturing methods. Modified release dosage forms include orally administered modified release dosage forms. Prolonged release (or extended release) dosage forms are modified release dosage forms that show a sustained release over a prolonged period of time. In delayed release dosage forms, release of the active substance is delayed for a certain period of time after administration or application of the dosage (the delay is also known as the lag time). The subsequent release can be similar to that of an immediate release dosage form. Multiphasic release dosage forms include biphasic release and pulsatile release. In biphasic release dosage forms, the first phase of drug release is determined by a fast release dose fraction providing a therapeutic drug level shortly after administration; and the second extended release phase provides the dose fraction required to maintain an effective therapeutic level for a prolonged period. Pulsatile drug release is intended to deliver a burst of drug release at specific time intervals. Multiple-unit: A multiple unit dosage form contains a plurality of units e.g. pellets or beads each containing release controlling excipients, e.g. in a gelatine capsule or compressed in a tablet. Single-unit: The single-unit dosage forms consist of only one unit, e.g. osmotic tablet.

Excipients and formulations for modified-release are well-known in the art and specific technologies are commercially available.

Suitably, pharmaceutical compositions of the invention are formulated to deliver 3-HB to the GI tract, preferably by oral administration. The human GI tract consists of digestive structures stretching from the mouth to the anus, including the oesophagus, stomach, and intestines. The GI tract does not include the accessory glandular organs such as the liver, biliary tract or pancreas. The intestines includes the small intestine and large intestine. The small intestine includes the duodenum, jejunum and ileum. The large intestine includes the cecum, colon, rectum and anus. The upper GI tract includes the buccal cavity, pharynx, oesophagus, stomach, and duodenum. The lower GI tract includes the small intestine below the duodenum and the large intestine. Preferably, the pharmaceutical compositions of the invention deliver the 3-HB to the lumen or mucosal surface of the GI tract, more preferably the lumen or mucosal surface of the large intestine, and more preferably the lumen or mucosal surface of the colon. Preferably, the pharmaceutical compositions of the invention deliver 3-HB to anaerobic sections of the lower GI tract, preferably the colon and/or terminal small intestine (ileum).

Various strategies have been proposed for targeting orally administered drugs to the colon, including: covalent linkage of a drug with a carrier, including those that enhance stability as well as increasing hydrophilicity; coating with pH-sensitive polymers; formulation of timed released systems; exploitation of carriers that are degraded specifically by colonic bacteria; bioadhesive systems; and osmotic controlled drug delivery systems. Various prodrugs have been developed that are aimed to deliver 5-aminosalicylic acid (5-ASA) for localized treatment of IBD. Microbially degradable polymers, especially azo-crosslinked polymers, have been investigated for use as coatings for drugs targeted to the colon. Certain plant polysaccharides such as amylose, inulin, pectin, and guar gum remain unaffected in the presence of gastrointestinal enzymes and have been explored as coatings for drugs for the formulation of colon-targeted drug delivery systems. Additionally, combinations of plant polysaccharides with crustacean extract, including chitosan or derivatives thereof, are proving of interest for the development of colonic delivery systems.

Examples of excipients for modified-release formulations include hydrogels that are able to swell rapidly in water and retain large volumes of water in their swollen structures. Different hydrogels can afford different drug release patterns and the use of hydrogels to facilitate colonic delivery has been investigated. For example, hydrogels and xerogels have been prepared using a high-viscosity acrylic resin gel, Eudispert hv, which has excellent staying properties in the lower part of the rectum over a long period. Eudragit® polymers (Evonik Industries) offer different forms of coating including gastro resistance, pH-controlled drug release, colon delivery, protection of and protection from actives.

Pharmaceutical compositions may be prepared according to any of the techniques known in the art, for example by mixing 3-HB, one or more pharmaceutically acceptable carrier, excipient and/or diluent and one or more modified-release excipient. Pharmaceutical compositions may be prepared by coating a core comprising 3-HB and one or more pharmaceutically acceptable carrier, excipient and/or diluent and optionally one or more modified-release excipient with a modified-release layer or coating using techniques in the art. For example, coatings may be formed by compression using any of the known press coaters. Alternatively, the pharmaceutical compositions may be prepared by granulation and agglomeration techniques, or built up using spray drying techniques, followed by drying.

Coating thickness can be controlled precisely by employing any of the aforementioned techniques. The skilled person can select the coating thickness as a means to obtain a desired lag time, and/or the desired rate at which drug substance is released after the lag time.

pH-dependent systems exploit the generally accepted view that pH of the human GI tract increases progressively from the stomach (where pH can be between about 1 and 2, which increases to pH 4 during digestion), through the small intestine (where pH can be between about 6 and 7) at the site of digestion, increasing in the distal ileum. Coating tablets, capsules or pellets with pH-sensitive polymers provides delayed release and protects the active drug from gastric fluid.

The pharmaceutical compositions of the invention can be formulated to deliver 3-HB to the GI tract at a particular pH. Commercially available excipients include Eudragit® polymers that can be used to deliver 3-HB at specific locations in the GI tract. For example, the pH in the duodenum can be above about 5.5. Eudragit® L 100-55 (Powder), Eudragit® L 30 D-55 (Aqueous dispersion), and/or Acryl-EZE® (Powder) can be used, for example as a ready-to-use enteric coating based on Eudragit® L 100-55. The pH in the jejunum can be from about 6 to about 7 and Eudragit® L 100 (Powder) and/or Eudragit® L 12,5 (Organic solution) can be used. Delivery to the colon can be achieved at a pH above about 7.0 and Eudragit® S 100 (Powder), Eudragit® S 12,5 (Organic solution), and/or Eudragit® FS 30 D (Aqueous dispersion) can be used. PlasACRYL™ T20 glidant and plasticizer premix, specifically designed for Eudragit® FS 30 D formulations can also be used.

The pharmaceutical compositions can be formulated to deliver the 3-HB at a pH of about 5 or more, such as about 5.1, 5.2, 5.3, 5.4, 5.5, 5.6, 5.7, 5.8 or 5.9 or more; preferably 6 or more, such as about 6.1, 6.2, 6.3, 6.4, 6.5, 6.6, 6.7, 6.8 or 6.9 or more; preferably 7 or more, such as about 7.1, 7.2, 7.3, 7.4, 7.5, 7.6, 7.8, 7.9 or 8. Preferably, the pharmaceutical compositions can be formulated to deliver the 3-HB at a pH of between about 5.5 and 7, between about 6 and 7.5, or between 7 and 8. In one embodiment, the pharmaceutical composition releases the 3-HB or 3-HB delivery means at the appropriate pH, thus delivering the 3-HB to the lumen of the GI tract, preferably to the terminal ileum and/or colon.

A pharmaceutical composition taken on an empty stomach is likely to arrive in the ascending colon about 5 hours after dosing, with the actual arrival dependent largely on the rate of gastric emptying. Drug delivery within the colon is greatly influenced by the rate of transit through this region. In healthy men, capsules and tablets pass through the colon in 20-30 hours on average. Solutions and particles usually spread extensively within the proximal colon and often disperse throughout the entire large intestine.

The pharmaceutical compositions of the invention can be formulated for time-controlled delivery to the GI tract, i.e. to deliver the 3-HB after a certain time (lag time) following administration.

Commercially available excipients for time-controlled delivery include Eudragit® RL PO (Powder), Eudragit® RL 100 (Granules), Eudragit® RL 30 D (Aqueous dispersion), and Eudragit® RL 12,5 (Organic solution). These excipients are insoluble, high permeability, pH-independent swelling excipients that can provide customized release profiles by combining with Eudragit® RS at different ratios. Eudragit® RS PO (Powder), Eudragit® RS 100 (Granules), Eudragit® RS 30 D (Aqueous dispersion), and Eudragit® RS 12,5 (Organic solution) are insoluble, low permeability, pH-independent swelling excipients that can provide customized release profiles by combining with Eudragit® RL at different ratios. Eudragit® NE 30 D (Aqueous dispersion), Eudragit® NE 40 D (Aqueous dispersion), and Eudragit® NM 30 D (Aqueous dispersion) are insoluble, low permeability, pH-independent swelling excipients that can be matrix formers.

Preferably, the pharmaceutical compositions can be formulated to deliver the 3-HB to the GI tract about 4 hours after administration. Preferably, the pharmaceutical compositions can be formulated to deliver the 3-HB between about 4 and 48 hours after administration, preferably between about 5 and 40 hours after administration, such as about 5, 10, 15, 20 or 24 hours after administration; preferably between about 5 and 10, 5 and 15, 5 and 20, or between about 10 and 24, 15 and 24 or 20 and 24 hours after administration. Preferably the pharmaceutical compositions are for administration between meals or with food, preferably with food. In one embodiment, the pharmaceutical composition releases 3-HB after the lag time. Alternatively, the pharmaceutical composition releases the 3-HB delivery means after the lag time.

Release of 3-HB or the 3-HB delivery means from the pharmaceutical compositions at the appropriate pH or after the lag time can be either immediate release or modified release. Immediate release and modified release formulations are known to those skilled in the art.

Release of the 3-HB or 3-HB delivery means from the pharmaceutical compositions can be measured by methods known in the pharmaceutical industry. Drug dissolution testing is routinely used to provide critical in vitro drug release information for both quality control purposes (to assess batch-to-batch consistency of solid oral dosage forms such as tablets) and drug development (to predict in vivo drug release profiles). Dissolution testing can be conducted in dissolution apparatus including USP Dissolution Apparatus 1—Basket (37° C.); USP Dissolution Apparatus 2—Paddle (37° C.); USP Dissolution Apparatus 3—Reciprocating Cylinder (37° C.); USP Dissolution Apparatus 4—Flow-Through Cell (37° C.).

Preferably, substantially no 3-HB is released from the pharmaceutical compositions until the appropriate pH is reached and/or until the lag time has expired. Preferably, substantially no 3-HB delivery means is released from the pharmaceutical compositions until the appropriate pH is reached and/or until the lag time has expired. Preferably not more than 10%/wt of the 3-HB or 3-HB delivery means is released from the pharmaceutical compositions, preferably not more than 9, 8, 7, 6, 5, 4, 3, 2 or 1%/wt of the 3-HB or 3-HB delivery means is released from the pharmaceutical compositions until the appropriate pH is reached and/or until the lag time has expired.

In a specific embodiment, pharmaceutical compositions can be formulated using Multi Matrix MMX® technology (Cosmo Pharmaceuticals Inc.), preferably as tablets. Tablets manufactured according to the MMX® technology are coated with pH-resistant acrylic copolymers which delay the release until the tablet reaches the indicated intestinal location where the programmed dissolution begins, thus protecting the active agents from adverse pH conditions and enzymatic presence in the upper GI tract. For example, pharmaceutical compositions can be formulated as Zacol NMX® (Cosmo Pharmaceuticals Inc.) tablets can include calcium 3-HB, Maltodextrin, Inulin, Sorbitol, Hypromellose, Microcrystalline Cellulose, Modified Corn starch, Citric Acid, Colloidal Silica Hydrate, Talc, Shellac, Magnesium Stearate, stearic Acid, Lecithin, Titanium Dioxide, Hydroxypropyl, Triethyl Citrate; Aroma: vanillin.

In another embodiment, pharmaceutical compositions can be formulated as a BioCare® capsule containing 3-HB buffered with calcium and magnesium (3-hydroxybutyric acid, calcium hydroxide, magnesium hydroxide and medium chain triglicerides), the capsule shell comprising hydroxypropyl methylcellulose, and comprising anti-caking agents silicon dioxide and magnesium stearate. Capsules are approximately 2.3 cm long.

Pharmaceutical compositions may be over-coated with a pharmaceutically acceptable film-coating, for aesthetic purposes (e.g. including a colourant), for stability purposes (e.g., coated with a moisture barrier), for taste-masking purposes, or for the purpose of protecting the 3-HB, prodrug, delivery system and/or excipients from aggressive media. Preferably, the pharmaceutical compositions can be over-coated with a gastro-protective or enteric coating, for example represented by a mixture of acrylic and/or methacrylic acid copolymers type A and/or type B (as, for example, Eudragit S100 and/or Eudragit L100). Preferably, the mixture of acrylic and/or methacrylic acid copolymers type A and/or type B is in a range ratio from 1:5 to 5:1. The gastro-protective coating also optionally comprises plasticizers, dyes, at least one water-solvent, at least one organic solvent or a mixture thereof.

The bacteria may be formulated with the usual excipients and components for such oral compositions, i.e., in particular fatty and/or aqueous components, humectants, thickeners, preservatives, texturing agents, flavour enhancers and/or coating agents, antioxidants, preservatives and/or dyes that are customary in the pharmaceutical and food supplement industry. Suitable pharmaceutically acceptable carriers include microcrystalline cellulose, mannitol, glucose, polyvinylpyrrolidone, and starch, or a combination thereof. The bacteria can then be formed into a suitable orally ingestible form. Suitable orally ingestible forms of probiotic bacteria can be prepared by methods well known in the pharmaceutical industry.

The composition to be administered orally may be formulated for example in the form of coated tablets, gel capsules, gels, emulsions, tablets, capsules, hydrogels, food bars, compact or loose powders, liquid suspensions or solutions, confectionery products, or food carriers. Preferably the composition is in a dry form. The preferred oral form for the composition is a solid form such as a capsule, tablet or powder.

The compositions may be formulated via the usual processes for producing oral formulations in particular coated tablets, gel capsules, gels, emulsions, tablets, capsules, hydrogels and powders.

The genetically engineered anaerobic bacteria that produce (R)-3-HB can also be incorporated as part of a food product, i.e. in yoghurt, milk or soy milk, or as a food supplement. Such food products and food supplements can be prepared by methods well known in the food and supplement industry.

The compositions can be incorporated into animal feed products as a feed additive.

By “delivering the 3-HB delivery means, the 3-HB or combination thereof” is meant that the 3-HB delivery means, the 3-HB or combination thereof is made available at a particular site in the subject such that 3-HB is absorbed and exhibits a therapeutic effect by increasing blood plasma levels of 3-HB to a therapeutically effective level. For example, 3-HB can be released from a 3-HB delivery system, such as anaerobic bacteria that produce the 3-HB in the colon; the 3-HB can be released from an oral dosage form, such as a capsule or tablet described herein that releases 3-HB in the colon; or the 3-HB can be delivered rectally.

The invention also encompasses methods of treating various diseases, disorders or conditions in a subject. The invention encompasses the use of the compositions described herein in the methods of treating various diseases, disorders or conditions described herein. Further, the invention encompasses the compositions described herein for use in the methods of treating various diseases, disorders or conditions described herein.

A method of treating comprises administering the pharmaceutical composition comprising the 3-HB delivery means, 3-HB or combination thereof to a subject for the purposes of ameliorating a disease, disorder or condition (i.e., slowing or arresting or reducing the development of the disease, disorder or condition or at least one of the clinical symptoms thereof); alleviating or ameliorating at least one physical parameter including those which may not be discernible by the patient; modulating the disease, disorder or condition, either physically (e.g., stabilization of a discernible symptom), physiologically (e.g., stabilization of a physical parameter), or both; or preventing or delaying the onset or development or progression of the disease or disorder or a clinical symptom thereof.

A subject is in need of a treatment if the subject would benefit biologically, medically or in quality of life from such treatment. Treatment will typically be carried out by a physician who will administer a therapeutically effective amount of the composition comprising the 3-HB delivery means, 3-HB or combination thereof. Suitably the subject is a human.

A therapeutically effective level or amount of 3-HB refers to an amount that will be effective for the treatments described herein, for example slowing, arresting, reducing or preventing the disease, disorder or condition or symptom thereof. Typically, a subject in need thereof is a subject presenting symptoms of the disease, disorder or condition. Alternatively, a subject may be susceptible to the disease, disorder or condition or has been tested positive for the disease, disorder or condition but has not yet shown symptoms.

The therapeutically effective amount of 3-HB depends on the 3-HB utilized (e.g. the ratio of (R)- to (S)-isomers), the subject being treated, the severity and type of the affliction, and the manner and route of administration.

Different organisms have different responses to fasting. In humans, baseline ketones (3-HB) rise from approximately 0.2 mM (after a switch from carbohydrate to fat utilisation as glycogen is fully depleted that occurs over a 12-72-hour period) up to about 5-7 mM where they stabilise (Longo, V. D. & Mattson, M. P. (2014) Cell Metab. 19, 181-192). Therapeutic effects associated with fasting, for example, have been observed for the diseases described herein.

Rahman, M. et al. (2014) Nat. Commun. 5, 3944 describes a mouse stroke model used to show that that the hydroxy-carboxylic acid receptor 2 (HCA2, GPR109A) is required for the neuroprotective effect of BHB and a ketogenic diet. 600-1,000 μM was found to be associated with neuroprotection.

3-HB has also been shown to increase histone acetylation in a dose dependent manner at 1 to 2 mM, which can occur in humans after a 2- to 3-day fast or strenuous exercise (Shimazu, T. et al. (2013) Science 339, 211-214).

2-10 mM concentrations of 3-HB in vitro reduced NLRP3 inflammasome activity (Youm, Y.-H. et al. (2015) Nat. Med. 21, 263-269).

A therapeutically effective amount of 3-HB in the blood can therefore be between about 0.2 mM to about 10 mM, preferably between about 0.5 mM and about 7 mM, preferably between about 0.5 mM and about 2 mM or between about 2 mM and about 5 mM, preferably about 2 mM or about 5 mM.

The inventors have surprisingly shown that by administering a pharmaceutical composition that releases 3-HB in the lower GI tract, preferably the colon, an increase in concentration of 3-HB in the blood plasma was observed (Example 8). A therapeutically effective amount of 3-HB can thus be delivered systemically by administering a pharmaceutical composition according to the invention. Specifically, after 5 days of dosing mice with 1.5×10⁸ spores/day of a genetically engineered strain of anaerobic bacteria that produce 3-HB as described herein, an increase from baseline 3-HB levels in mouse blood serum of 100 μM was observed. An increase in 3-HB concentration in the colon of 200 μM was observed.

The human colon volume (sum of ascending/descending and transverse) is around 600 ml (Pritchard, S. E. et al. (2-14) Neurogastroenterol. Motil. 26, 124-130) whereas the entire intestine of a mouse is around 1 ml in volume (McConnell, E. L., Basit, A. W. & Murdan, S. (2008) J. Pharm. Pharmacol. 60, 63-70). The approximate total GI transit time is around 5-6 hours in a mouse (Padmanabhan, P., et al. (2013) EJNMMI Res. 3, 60 and Kashyap, P. C. et al. (2013) Gastroenterology 144, 967-977) and the colon transit times have been estimated to be between 23 and 40 hours in humans (Degen, L. P. & Phillips, S. F. (1996) Gut 39, 299-305 and Wagener, S., et al (2004) J. Pediatr. Surg. 39, 166-169-169). Since transit time in the human gut is five times longer than in mouse, fewer spores are needed (by a factor of five) to achieve the same concentration of 3-HB if the colon volumes were the same. Further, because the bacteria are resident in the human colon approximately five time longer than the mouse colon, there will be a longer duration for cell division (by a factor of five), therefore resulting in more cell numbers and in an increase in production of 3-HB. The lab fermentation based doubling time of the bacterial strain CHN1 is similar to that for E. coli and E. coli have a gut doubling time of about 3 hours (Myhrvold, C., et al (2015) Nat. Commun. 6, 10039). CHN1 will undergo 10 doublings of cells during gut transit, equating to a three order of magnitude increase in cell numbers. In the mouse there is only sufficient time for around two doublings of cells equating to less than a 10-fold increase in cell numbers. Approximately 100 times more cells will grow from each spore delivered to the human gut relative to the mouse gut. When accounting for gut volume differences, colon transit times and cell division within the gut, approximately the same dose delivered to a mouse and a human will result in approximately the same concentration of 3-HB within the gut lumen.

To deliver a therapeutically effective amount of 3-HB systemically in humans using a composition according to the invention, the number of spores is increased from 1.5×10⁸ to about 1.5×10⁹ spores per day. Further, an increase in yield/cell is achieved using a plasmid bearing strain and following integration of the relevant genes into the chromosome as described herein (see Example 9) and a 5-fold increase in 3-HB yield is achieved. Therefore, using the same dosing regimen from the mouse experiment a 5 mM increase in 3-HB in the blood can be achieved in humans. The dosage can be adjusted according to therapeutically effective concentrations achieved by persons skilled in the art.

The dosage regimen can be adjusted by those skilled in the art to achieve the appropriate delivery of 3-HB and therapeutically effective blood concentrations of 3-HB. Suitable dosage regimens can be administration of from about 1×10⁸ spores per day to about 1×10¹¹ spores per day. Dosing can be continued as necessary to achieve appropriate 3-HB levels in the blood. Preferably, about 1×10⁸ spores per day to about 1×10¹¹ spores per day can be administered for up to five days, preferably from about 1×10⁹ spores per day to about 1×10¹⁰ spores per day for five days, preferably about 1.5×10⁹ spores per day for five days. Dosing can be continued as necessary to maintain appropriate 3-HB levels in the blood. The appropriate dose can be administered in a single tablet, for example, or in multiple tablets.

A therapeutically effective amount of 3-HB in the blood can be achieved by administering a composition according to the invention such that between about 200 μM and 20 mM 3-HB is delivered to the gut lumen, preferably between about 5 mM and 10 mM, preferably about 10 mM. The dosage can be adjusted according to therapeutically effective concentrations achieved by persons skilled in the art.

A dose can be administered as part of a meal or snack or liquid, wherein the subject is provided with a dry dose for mixing with or combining with the meal, snack or liquid (for example water or fruit juice).

In accordance with the invention, the 3-HB delivery means, 3-HB or combination thereof can be administered in combination with one or more additional therapeutic agents.

Administration includes administration of a formulation that includes the 3-HB delivery means, 3-HB or combination thereof and one or more additional therapeutic agents, or the essentially simultaneous, sequential or separate administration of separate formulations of the 3-HB delivery means, 3-HB or combination thereof and one or more additional therapeutic agents. In one embodiment, the 3-HB delivery means also delivers one or more additional therapeutic agents to the lower GI tract.

Compositions of the invention can also be administered in conjunction with fasting, a ketogenic diet, and/or administration of exogenous ketones.

The invention encompasses methods of treating a disease, disorder or condition in a subject using a pharmaceutical composition as described herein. In one embodiment, the disease disorder or condition is characterised by oxidative stress. 3HB and a ketogenic diet have been shown to counter oxidative stress by inhibition of histone deacetylase (HDAC) enzymes. Diseases include recovery from spinal cord injury, neurodegenerative diseases including Alzheimer's disease (AD), Parkinson's disease (PD), aging and other neural disorders including amyotrophic lateral sclerosis (ALS). In another embodiment, the disease disorder or condition is mediated by the NLRP3 inflammasome. Diseases include type 2 diabetes, atherosclerosis, multiple sclerosis, AD, age-related functional decline, bone loss and gout. In another embodiment, the disease disorder or condition is HCA2 receptor-mediated. Diseases include atherosclerosis, obesity, diabetic retinopathy, neurodegenerative diseases, neurologic disease conditions including juvenile epilepsy, AD, ALS, HD, PD, multiple sclerosis, ischemic stroke and traumatic brain injury. In another embodiment, the disease disorder or condition is modulated by a ketogenic diet and or fasting. Diseases include epilepsy, in particular childhood drug refractory epilepsy; cancer, in particular Glioblastoma; type 2 diabetes; Neurodegenerative diseases such as PD and AD; nervous system and/or metabolic dysregulation.

In one embodiment, the invention encompasses methods of treating a metabolic disease, disorder or condition in a subject. The metabolic disease disorder or condition can be any metabolic disease known to those skilled in the art that has been shown to respond to elevated 3-HB levels in the blood, for example as a result of fasting, a ketogenic diet and/or by administration of exogenous 3-HB. The metabolic disease disorder or condition can be selected from diabetes, obesity, metabolic syndrome and related diseases such as diabetic retinopathy and NAFLD/NASH. Preferably, the metabolic disease disorder or condition is type 2 diabetes.

In another embodiment, the invention encompasses methods of treating a neurodegenerative disease, disorder or condition in a subject. The neurodegenerative disease disorder or condition can be any neurodegenerative disease known to those skilled in the art that has been shown to respond to elevated 3-HB levels in the blood, for example as a result of fasting, a ketogenic diet and/or by administration of exogenous 3-HB. The neurodegenerative disease disorder or condition can be selected from AD, PD, HD, ALS and multiple sclerosis. Preferably, the neurodegenerative disease disorder or condition is AD, PD or HD.

In another embodiment, the invention encompasses methods of treating a neurological disease, disorder or condition in a subject. The neurological disease disorder or condition can be any neurological disease known to those skilled in the art that has been shown to respond to elevated 3-HB levels in the blood, for example as a result of fasting, a ketogenic diet and/or by administration of exogenous 3-HB. The neurological disease disorder or condition can be selected from epilepsy, brain injury and stroke. Preferably, the neurological disease disorder or condition is ischemic stroke or juvenile epilepsy.

In another embodiment, the invention encompasses methods of treating an inflammatory or autoimmune disease, disorder or condition in a subject. The inflammatory or autoimmune disease disorder or condition can be any inflammatory or autoimmune disease known to those skilled in the art that has been shown to respond to elevated 3-HB levels in the blood, for example as a result of fasting, a ketogenic diet and/or by administration of exogenous 3-HB. The inflammatory or autoimmune disease disorder or condition can be selected from atherosclerosis, multiple sclerosis, pancreatitis, sepsis, psoriasis, gout, diabetic retinopathy, diabetic nephropathy, rheumatoid arthritis, psoriatic arthritis, arthritis, ankylosing spondylitis, COPD or neuro-inflammation. Preferably, the inflammatory or autoimmune disease disorder or condition is atherosclerosis, neuro-inflammation, diabetic retinopathy or pancreatitis. In certain embodiments, the inflammatory or autoimmune disease disorder or condition is not Crohn's disease, ulcerative colitis, pouchitis, collagenous colitis and lymphocytic colitis, colorectal cancer, rheumatoid arthritis, multiple sclerosis, psoriasis, psoriatic arthritis, gout, ankylosing spondylitis or COPD. In certain embodiments, the inflammatory or autoimmune disease, disorder or condition is not an inflammatory or autoimmune disease, disorder or condition of the GI tract, preferably not of the large intestine, preferably not of the colon. In certain embodiments, the inflammatory or autoimmune disease is not an IBD, preferably not Crohn's disease, ulcerative colitis, or colorectal cancer.

In another embodiment, the invention encompasses methods of treating cancer in a subject. The cancer can be any cancer known to those skilled in the art that has been shown to respond to elevated 3-HB levels in the blood, for example as a result of fasting, a ketogenic diet and/or by administration of exogenous 3-HB. The cancer can be selected from lymphoma, melanoma, neuroblastoma, glioblastoma, kidney, thyroid, pancreas and breast cancer. In certain embodiments, the cancer is not colorectal cancer.

The growth and degree of colonisation in the gut of the genetically engineered bacteria can be controlled by species and strain choice and/or by providing specific food that the bacteria thrive on as a prebiotic, either within the same dose that contains the probiotic or as a separately ingested composition.

The composition may also further comprise a prebiotic to enhance the growth of the administered probiotic. The prebiotic may be administered sequentially, simultaneously or separately with a composition comprising genetically engineered anaerobic bacteria that produce (R)-3-HB. The prebiotic and genetically engineered bacteria can be formulated together into the same composition for simultaneous administration. Alternatively, the bacteria and prebiotic can be formulated separately for simultaneous or sequential administration.

Prebiotics are substances that promote the growth of probiotics in the intestines. They are food substances that are fermented in the intestine by the bacteria. The addition of a prebiotic provides a medium that can promote the growth of the probiotic strains in the intestines. One or more monosaccharides, oligosaccharides, polysaccharides, or other prebiotics that enhances the growth of the bacteria may be used.

Preferably, the prebiotic may be selected from the group comprising of oligosaccharides, optionally containing fructose, galactose, mannose; dietary fibres, in particular soluble fibres, soy fibres; inulin; or combinations thereof. Preferred prebiotics are fructo-oligosaccharides (FOS), galacto-oligosaccharides (GOS), isomalto-oligosaccharides, xylo-oligosaccharides, oligosaccharides of soy, glycosylsucrose (GS), lactosucrose (LS), lactulose (LA), palatinose-oligosaccharides (PAO), malto-oligosaccharides, pectins, hydrolysates thereof or combinations thereof.

All of the features described herein (including any accompanying claims, abstract and drawings), and/or all of the steps of any method so disclosed, may be combined with any of the above aspects in any combination, except combinations where at least some of such features and/or steps are mutually exclusive. Specifically, any of the active agents and compositions described herein can be used in any of the described methods of treatment. Any and all such combinations are explicitly envisaged as forming part of the invention.

### EXAMPLES

The invention will now be explained in further detail with reference to the following Examples.

### Example 1

### Production of (R)-3-HB in C. Butyricum Expressing phaB

1) Gene Synthesis

The gene Cupriavidus necator phaB was codon optimised for Clostridia. FIG. 2 shows one example of the codon optimised sequence which was synthesized by Gene Art® (Thermo Fisher Scientific).

2) Plasmid Assembly

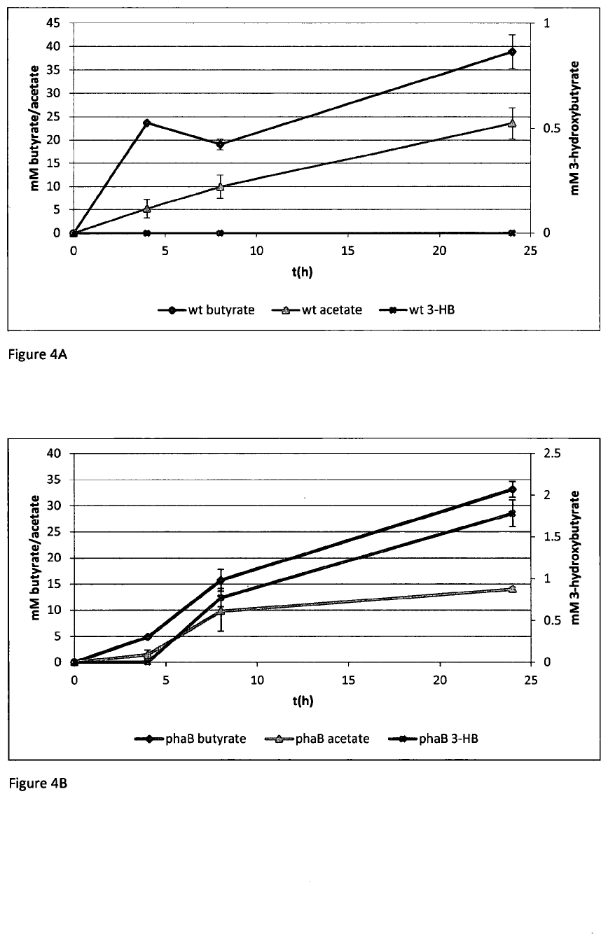

phaB was cloned into plasmid pMTL83251 under control of the C. sporogenes Pfdx promoter using standard cloning techniques yielding plasmid pMTL83251_pfdx_phaB (see FIG. 3)

3) Strain Development

Plasmid pMTL83251_pfdx_phaB was conjugated into Clostridium butyricum ATCC19398/DSM10702 using E. coli CA434 as conjugation donor. A strain specific conjugation protocol was applied. Briefly, overnight cultures of E. coli CA434 carrying plasmid pMTL83251_pfdx_phaB and C. butyricum were used to inoculate 9 ml LB and RC broth respectively. Cultures were grown until OD₆₀₀ of 0.5-0.7 was reached. 1 ml of E. coli culture was spun down and the pellet mixed with 200 μl heat-shocked (50° C. 10 min) C. butyricum culture. The cell mix was spotted on a non-selective RCM plate and incubated overnight. The incubated mix was re-suspended into 500 μl fresh RCM and plated on selective media containing 10 μg/ml erythromycin. Presence of the plasmid within the obtained transconjugants was confirmed by PCR using plasmid specific primers.

4) Fermentation Data for C. Butyricum

Growth Method

RC broth containing per 1 L: yeast extract 13 g, Peptone 10 g, soluble starch 1 g, sodium chloride 5 g, sodium acetate 3 g, cysteine hydrochloride 0.5 g, carbohydrate 2%, was used. Calcium carbonate 10 g/L was added to liquid culture for pH regulation. Solid media contained 15 g/L agar.

Transformants were grown overnight in seed cultures (RC broth) at 37° C. 100 ml RC broth containing 2% glucose was inoculated to a starting OD of 0.05-0.1. Strains were grown anaerobically at 37° C. in the presence of required antibiotic. Samples for metabolic analysis were taken at regular intervals.

Analysis and Results

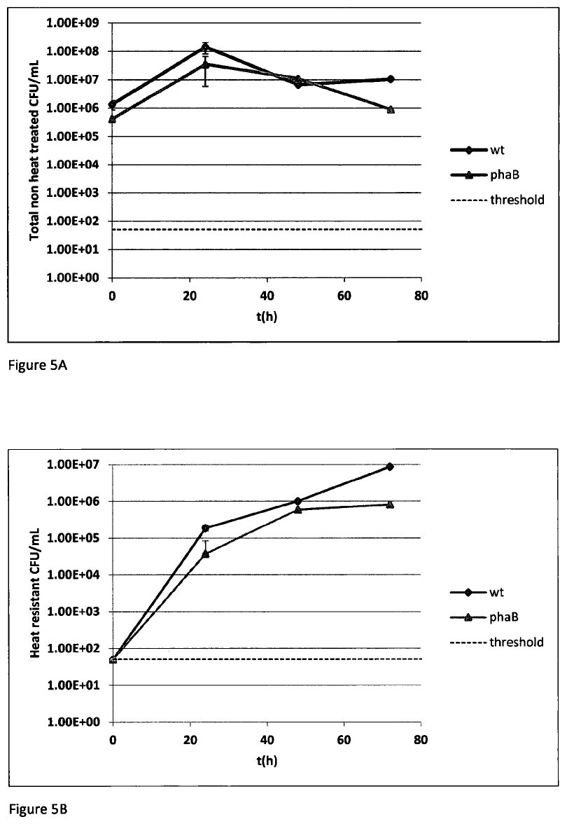

Culture supernatant of the engineered C. butyricum (CHN-1) was analysed for (R)-3-HB and (5)-3-HB using the 3-HB assay kit (Sigma Aldrich). The strain expressing phaB produced only (R)-3-HB. Culture supernatants of CHN-1 and a native C. butyricum were also analysed for production of SCFAs and (R)-3-HB using HPLC-RI. The phaB expression strain of C. butyricum (CHN-1) produced about 187 mg/L after 24 h growth as shown in FIG. 4B. The wildtype C. butyricum strain only produced butyrate and acetate as shown in FIG. 4A.

### Example 2

### Formulations for Colonic Delivery

Zacol NMX® is a dietary supplement (nutraceutical) based on the MMX® technology and directed to the colon. It is a product based on the application of MMX® technology to a combination of calcium salt of butyric acid and inulin. NMX® is a nutraceutical version of MMX® technology. Tablets contain calcium 3-HB (0.307 g), Maltodextrin, Inulin (0.250 g), Sorbitol, Hypromellose, Microcrystalline Cellulose, Modified Corn starch, Citric Acid, Colloidal Silica Hydrate, Talc, Shellac, Magnesium Stearate, stearic Acid, Lecithin, Titanium Dioxide, Hydroxypropyl, Triethyl Citrate; Aroma: vanillin.

BioCare format. Capsules contain 1815 mg 3-HB, 243 mg calcium hydroxide, 123 mg magnesium hydroxide, medium chain triglycerides, capsule shell (hydroxypropyl methylcellulose), anti-caking agents (silicon dioxide & magnesium stearate). One capsule is taken three times a day with food, or as professionally directed.

### Example 3

### Bacterial Delivery

Spore Formation of C. Butyricum

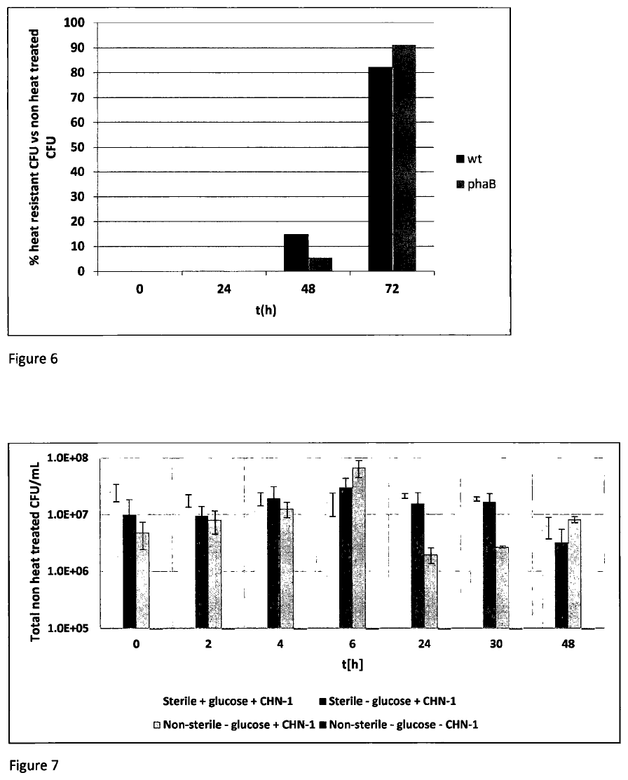

The same culture medium and inoculation techniques as for fermentation were used. Samples were taken at the start of the experiment and in regular intervals for 72 h to determine the ratio of vegetative cells to spores. For enumeration of spores, samples were heat treated at 65° C. for 30 min to kill any vegetative cells. Simultaneously, samples taken for enumeration of total CFU count (vegetative cells+spores), were placed on the bench to prevent further growth in the medium. Heat treated and non-heat treated samples were then serially diluted and plated in 20 μL discreet spots in triplicate on non-selective medium for wildtype and on selective medium for engineered strains. After overnight incubation at 37° C. anaerobically, CFU/mL were determined. FIGS. 5A-B show the development of spores over 72 hours. FIG. 6 shows the percentage of spores in total CFU in the culture over 72 hours.

Tablet formulations includes corn starch, lactose, hydrated magnesium silicate, microcrystalline cellulose, magnesium stearate and sucrose.

### Example 4

### Evaluation of CHN-1 in a Simulated Colon Environment

Spores of engineered C. butyricum (CHN-1) were produced using pH controlled laboratory scale bioreactors. Strains were handled in nitrogen and carbon dioxide flushed anaerobic workstations at 37° C. before inoculation into bioreactors.

CHN-1 was grown on Reinforced Clostridial agar (Sigma-Aldrich, UK) plates from spore stocks. A single colony was used to inoculate modified Reinforced Clostridial (RC) broth (per litre: Yeast 13 g, Peptone 10 g, Starch 1 g, NaCl 5 g, CH₃COONa 3 g, Cysteine hydrochloride 0.5 g, CaCO₃ 10 g, Glucose 20 g), which was then serially diluted 10⁰-10⁻⁸ in modified RC broth. After 8-12 hours, a 1:10 dilution was prepared in fresh modified RC from highest diluted o/n culture grown (usually 10⁻⁶) into a day culture. The day culture was typically grown 1½-2 hours before it was transferred into serum bottles. Serum bottles were capped with a rubber stopper to maintain anaerobiosis. Bacterial culture from the serum bottles was used to inoculate the bioreactor 1:10. Bioreactors contained modified RC, were pH controlled at 6.5 using 3M sterile KOH as required, with 125 rpm agitation and 6 L/h N₂ flushing. Bioreactors were maintained at 37° C. throughout. Cell mass was harvested after 24 hours and stored at 4° C. before purification. Vegetative cells were disrupted by heat treatment at 65° C. Purification entailed repeated washing steps using sterile deionised water with centrifugation at 5000×g for 20 min. Spores were enumerated using an improved Neubauer counting chamber and viable spore count was assessed by colony forming units on RC agar.

The capacity of CHN-1 spores to germinate and grow in the colon environment was assessed using a simulation of the proximal large intestine as described by Molly et al., (1993) Appl Microbiol Biotechnol. Pre-reduced sugar-depleted base colon medium containing nutrients that are present in the colon (e.g. host or diet derived glycans such as mucin or starch) was added to double-jacketed glass bioreactors. CHN-1 spores and/or faecal inoculum were added to bioreactors inside the anaerobic workstation. The faecal inoculum was prepared from faecal donor material of a single healthy donor by mixing fresh faecal sample 1:5 with pre-reduced phosphate buffer and removal of particles by centrifugation at 500×g. The inoculum was then added to the bioreactor at dilution of 1:10. Bioreactors were sealed with rubber stoppers to maintain anaerobiosis.

The experiment entailed 4 different conditions in triplicate: i) inoculation with filter-sterilized faecal suspension and CHN-1; ii) inoculation with filter-sterilized faecal suspension, CHN-1 and glucose (1 g/L); iii) inoculation with faecal suspension and CHN-1; and iv) inoculation with faecal suspension. Bioreactors were maintained at 37° C. and continuous mixing was applied at 90 rpm. Samples were removed for analysis at t=0, 2, 4, 6, 24, 30, and 48 hours after inoculation.

Germination of spores, growth and metabolic activity of CHN-1 was assessed by 1) colony forming units on selective medium; 2) pH decrease; 3) SCFA production; and 4) production of (R)-3-HB. 5)
Detection of CHN-1 was performed using two specific PCR protocols for detection of C. butyricum 16s-23s intergenic spacer region and detection of phaB.

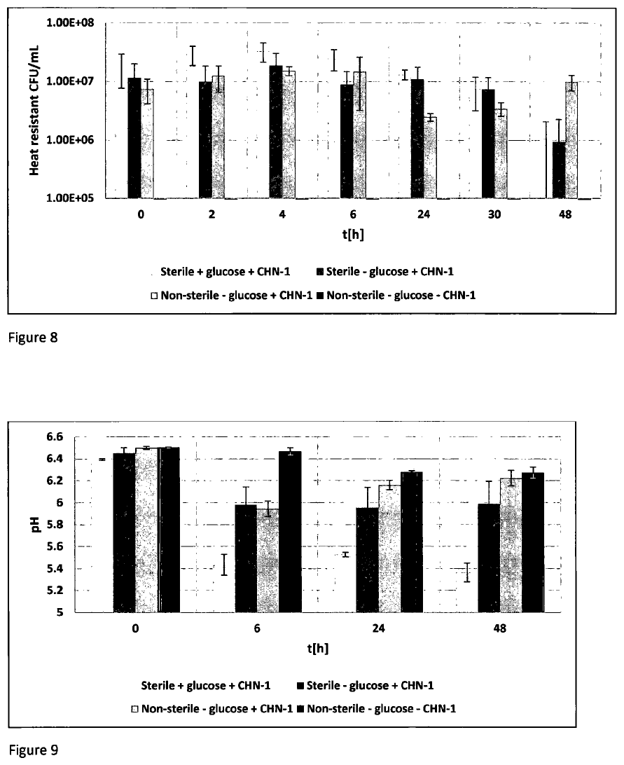

1) Colony forming units were assessed on modified C. butyricum isolation medium (BIM) as described by Popoff (1984) J Clin Microbiol, using D-Cycloserine as sole antibiotic agent at a concentration of 250 μg/mL. This medium allowed for the selective enumeration of CHN-1 with no colonies being observed in background without supplementation of this strain (detection threshold 200 CFU/mL). Total viable counts (FIG. 7) were assessed by serially diluting samples and plating onto modified BIM. Colony forming units were enumerated after overnight incubation in anaerobic conditions. Heat-resistant counts (FIG. 8) were assessed by pasteurizing samples for 30 min at 65° C. before serially diluting and plating onto modified BIM. Colony forming units were enumerated after overnight incubation in anaerobic conditions.

As shown in FIG. 7, there was no significant difference between the total viable count enumerated from reactors inoculated with filter-sterilized faecal suspension and CHN-1 with or without glucose. In bioreactors inoculated with CHN-1 and faecal suspension there was a steady increase of total viable count for the first 6 hours of the experiment, indicating germination and growth of CHN-1. There was a statistically significant higher number of total viable count of CHN-1 in bioreactors inoculated with filter-sterilized faecal suspensions compared to those inoculated with faecal suspension after 24 hours (p<0.05), indicating that there was competition between the faecal microbiota and CHN-1 in those bioreactors. The bioreactors not inoculated with CHN-1 did not return any CFU above the detection threshold.

As shown in FIG. 8, there was no significant difference in heat-resistant count enumerated from bioreactors inoculated with filter-sterilized faecal suspension and CHN-1 with or without glucose. There was a steady decrease in heat-resistant count in all bioreactors between 4 and 24 hours of incubation, indicating the germination and outgrowth from spores leading to vegetative cell growth.

2) The decrease in pH (FIG. 9) caused by metabolic activity within the colonic simulation was measured at t=0, 6, 24, and 48 hours using a Senseline F410 pH meter (ProSense, Oosterhout, NL).

As shown in FIG. 9, there was no significant difference in the pH of the four experimental set-ups at the start of the experiment. Nor was there a significant difference in the pH of the bioreactors inoculated with filter-sterilized faecal inoculum and CHN-1, and the bioreactors seeded with faecal suspension and CHN-1 at any time during the experiment. There was, however, statistically significant difference in pH of the bioreactors inoculated with filter-sterilized faecal suspension and CHN-1 when comparing those with added glucose to those without from time point 6 hours onwards (p<0.02). There was also statistically significant difference between the bioreactors inoculated with faecal suspensions when comparing those with addition of CHN-1 and those without at time points 6 hours and 24 hours (p=0.003 and 0.0133, respectively). This indicates that CHN-1 is metabolically active in each of the respective backgrounds, with filter-sterilized faecal suspension and with faecal suspension within the first 6 hours of instillation.

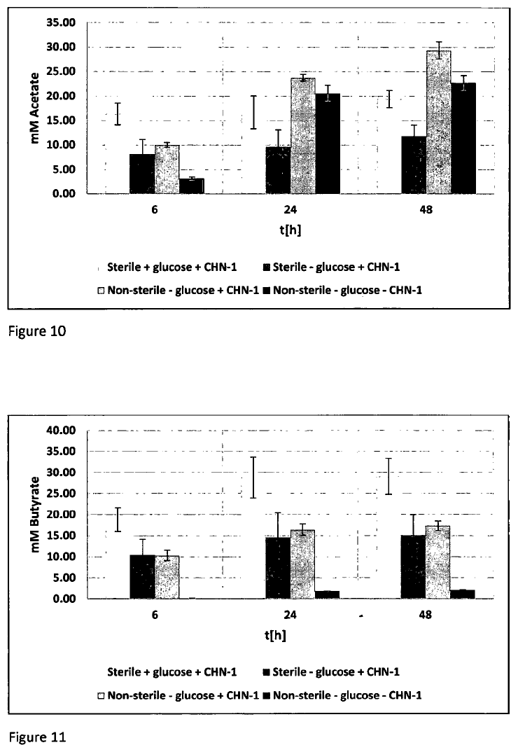

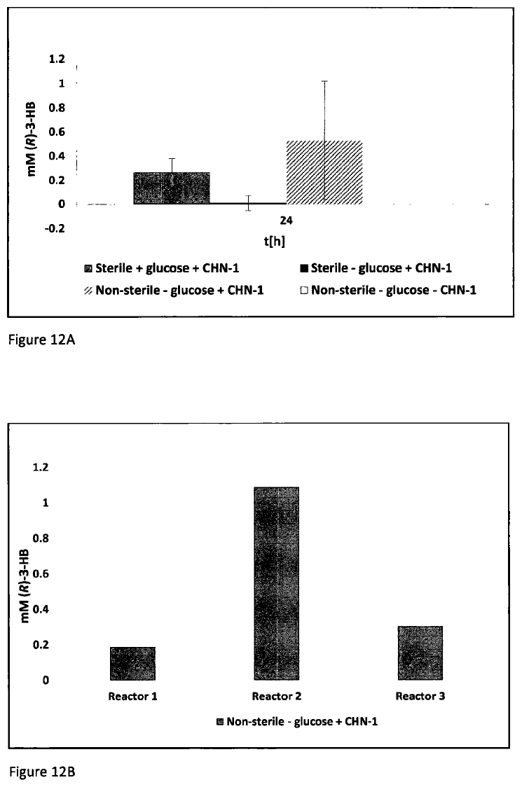

3) The production of SCFA acetate (FIG. 10), and butyrate (FIG. 11) was measured by gas chromatography as described by De Weirdt et al. (2010) FEMS Microbiol Ecol.

As shown in FIG. 10, there was a statistically significant increase in acetate concentration in bioreactors dosed with faecal inoculum and CHN-1 compared to those inoculated with faecal inoculum only after 6 hours of incubation until the end of the experiment (p<0.04). There was also a significantly higher amount of acetate in the bioreactors containing faecal inoculum and CHN-1 compared to those containing filter-sterilized faecal suspension and CHN-1 from time point 24 hours onwards (p<0.003).

As shown in FIG. 11, there was a statistically significant increase in butyrate concentration in bioreactors dosed with faecal inoculum and CHN-1 compared to those dosed with faecal suspension only throughout the experiment (p=0.0001). There was no significant difference between the bioreactors containing CHN-1 with or without faecal suspension. The highest amount of butyrate was found in those reactors, where glucose had been supplemented as a precursor of butyrate production (p<0.04).

4) The production of (R)-3-HB in the colonic simulation was measured by HPLC-RI using an Aminex HPX-87H column of 300 mm×7.8 mm with 9 μm particle size (Biorad, USA) on a Dionex UltiMate 3000 System (Thermo Scientific, USA) set to 35° C. and a run time of 55 min. As mobile phase, 5 mM H₂SO₄ with a flow rate of 0.5 mL/min was used. Culture samples were filter-sterilized prior to analysis using KX syringe filters of 13 mm diameter with regular cellulose of pore size 0.22 μm (Kinesis Ltd, UK). A known amount of (R)-3-HB was spiked into samples before samples were mixed 1:1 with mobile phase containing 50 mM valerate as internal standard. Calibration standards containing increasing concentrations of glucose, (R)-3-HB, butyrate, acetate, and lactate were run at the same time as the spiked samples.

As shown in FIG. 12A, there was a significantly higher amount of (R)-3-HB produced in the bioreactor inoculated with filter-sterilized faecal suspension, CHN-1 and glucose in comparison to the bioreactor without glucose. There was a significantly higher amount of (R)-3-HB present in any bioreactor dosed with CHN-1 compared to those dosed with faecal suspension only (p<0.03). This indicates the production of (R)-3-HB in all bioreactors is dependent on the presence of CHN-1.

FIG. 12B shows the (R)-3-HB concentrations measured for each of the three experimental replicates conducted for the third experimental condition (inoculation with faecal suspension and CHN-1) of FIG. 12A. These three values are combined and represented in FIG. 12A. (R)-3-HB was detected in all three replicates above the baseline concentrations found in human blood serum (0-200 μM) under fed conditions. One reactor showed levels at around 1 mM. This range of 200 μM-1 mM (R)-3-HB was found to be effective at reducing the expression of multiple inflammatory proteins and increasing the expression of anti-inflammatory proteins in two human colon tissue based in vitro models, as described in Examples 5 and 6.

5) The presence of CHN-1 in the bioreactors was further confirmed using strain-specific PCR. For this, two different sets of oligonucleotides were used (table 1). One set amplified the 16s-23s intergenic spacer region of C. butyricum as described by Nakanishi et al. (2005) Microbiol Immunol. The second set specifically amplified the integrated phaB gene.

TABLE 1
Oligonucleotide sequences

| | | SEQ |
|---|---|---|
| Name | Sequence | ID NO. |
| ISR-F | CCTCCTTTCTATGGAGAAATCTAGCA | 2 |
| ISR-R | TGTAGCTTGACCTTTTTAAGTTTTGA | 3 |
| phaB-F | GTGTAGTAGCCTGTGAAATAAG | 4 |
| phaB-R | GAGGCACATTTATTTTAGCTAGCTTA | 5 |
| | CTAACCCATGTG | |

Genomic DNA was extracted using phenol-chloroform extraction before subjected to PCR using both oligonucleotide sets.

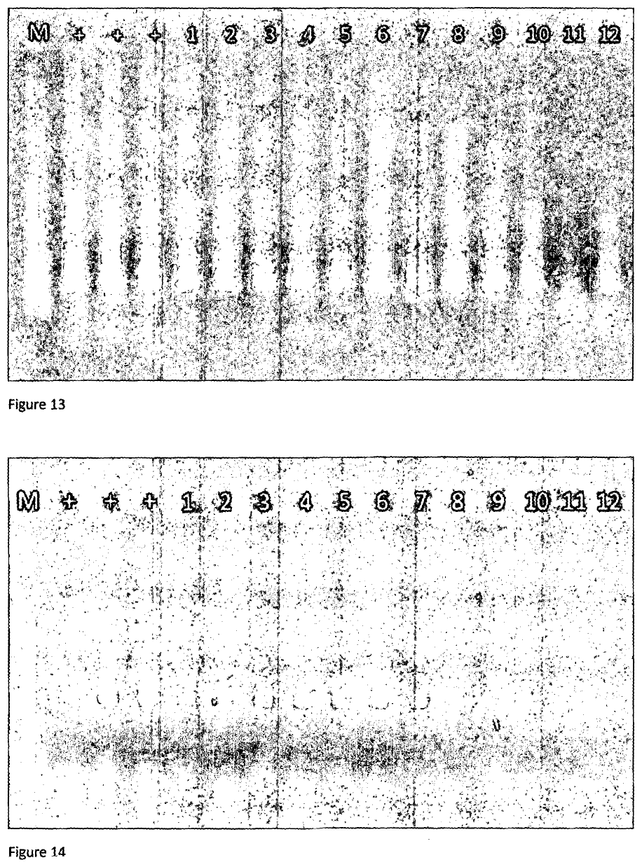

As shown in FIG. 13, the presence of C. butyricum was confirmed in samples 1-9, corresponding to bioreactors dosed with CHN-1 spores. Low levels of C. butyricum (below detection level in total viable count and heat-resistant count (FIGS. 7 and 8)) were also confirmed in reactors 10-12, which were dosed with faecal suspension only.

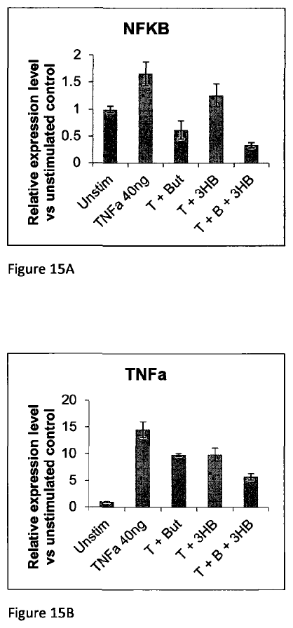

As shown in FIG. 14, the presence of phaB was confirmed in samples 1-9, corresponding to bioreactors dosed with CHN-1 spores. No amplicon was observed in reactors dosed with faecal suspension only, confirming absence of phaB in these bioreactors.

In summary, these data show that CHN-1 spores can germinate in the simulated colonic environment. Germination of these spores subsequently leads to vegetative cell growth as shown by indicators of metabolic activity, here SOFA concentration and pH drop. The data also shows that CHN-1 successfully introduces a new metabolic product, namely (R)-3-HB, which is not normally found in the colonic environment.

### Example 5

### Modelling Inflammation Using Primary Human Intestinal Organoids

To measure the effect of (R)-3-HB on inflammation of primary human intestinal organoids, healthy large intestine samples of a female 67-year old donor were cultured for 8 weeks in vitro prior to experiments. Method as used in Hannan et al., Stem Cell Reports. Vol. 1, 293-306 Oct. 15, 2013.

1) To induce inflammation, organoids were treated with TNFα (40 ng/mL) alone or in combination with butyrate (10 μM), (R)-3-HB (10 μM), or butyrate and (R)-3-HB for 18 hours. Subsequently, cells were lysed and RNA was isolated for cDNA synthesis and measurement of mRNA expression levels of inflammation factors, NF-κB and TNFα, by qPCR.

FIG. 15 shows the relative mRNA expression levels, as standardised against the control (unstimulated sample) set to 1, of inflammatory factors, NF-κB (A) and TNFα (B) expressed by primary human intestinal organoids in response to incubation with TNFα in combination with butyrate, (R)-3-HB, or a combination of butyrate with (R)-3-HB. The mRNA expression of the inflammation factors NF-κB and TNF-α decreased with treatment with butyrate, (R)-3-HB, or a combination of butyrate and (R)-3-HB.

2) To induce inflammation, organoids were treated with TNF-α (40 ng/mL) alone or in combination with (R)-3-HB (sodium salts at 10 μM) for 18 hours. Subsequently, cells were lysed and RNA was isolated for cDNA synthesis and mRNA expression levels of a panel of inflammation factors was measured by qPCR.

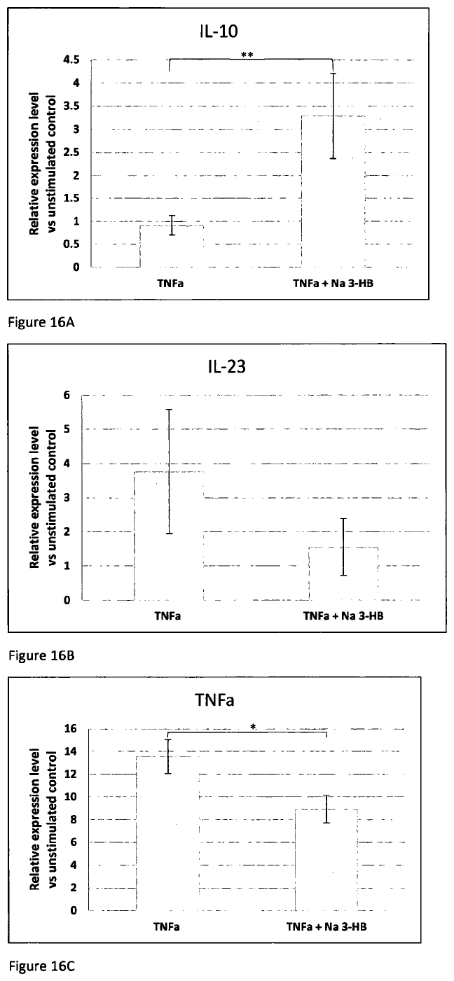

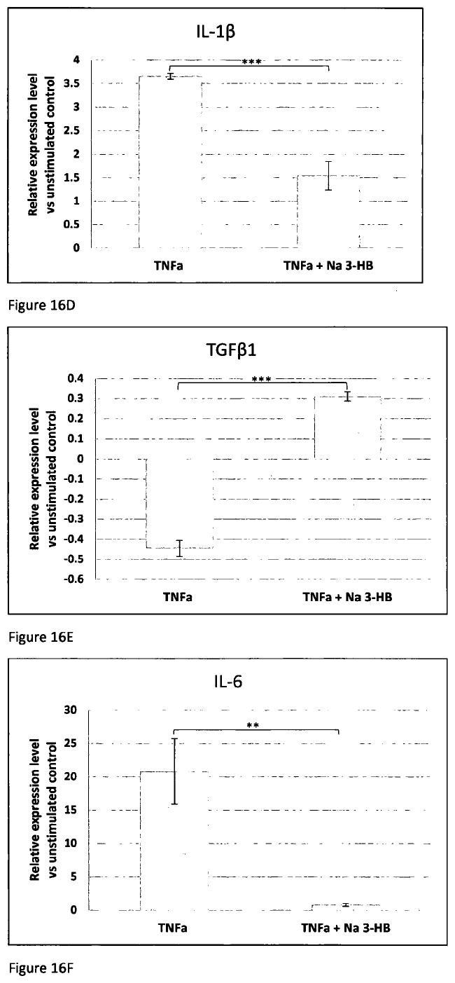

FIG. 16 shows the relative mRNA expression levels, as standardised against the normalized control (unstimulated sample set to 0), of inflammatory factors, expressed by primary human intestinal organoids in response to incubation with TNF-α alone or in combination with (R)-3-HB. The mRNA expression of pro-inflammatory cytokines and proteins IL-23, TNF-α, IL-1β, IL-6 and NF-κβ decreased in the presence of (R)-3-HB compared to treatment with TNF-α alone. The mRNA expression of anti-inflammatory cytokines TGF-β1 and IL-10 increased in the presence of (R)-3-HB compared to treatment with TNF-α alone.

Both butyrate and (R)-3-HB act on pro-inflammatory cytokines and proteins and anti-inflammatory cytokines and proteins. (R)-3-HB has greater reducing effect than butyrate on multiple important pro-inflammatory regulators of IBD and greater inducing effect on major protective regulators of intestinal inflammation (data not shown).

3) The effect of (R)-3-HB and the related SCFA butyrate on the inflammatory response exerted by primary human intestinal organoids upon co-incubation with TNF-α as inflammatory stimulus was measured. Primary human intestinal organoids were cultured for 8 weeks prior to experiments as described in Hannan et al. (2013) Stem Cell Reports.

To induce inflammation, organoids were treated with 60 ng/mL TNF-α alone or in combination with different concentrations of the sodium salts of (R)-3-HB or butyrate for 24 hours. Subsequently, cells were lysed and RNA was isolated using the RNeasy mini kit (Qiagen Ltd, Germany) for synthesis of cDNA. mRNA expression levels of IL-23 was measured by qPCR using the SensiMix SYBR low-ROX kit (Bioline, UK).

Values were normalized versus untreated control and graphs show the difference of mRNA levels measured in organoids treated with TNF-α alone (set as 0) and those treated with TNF-α and butyrate or (R)-3-HB.

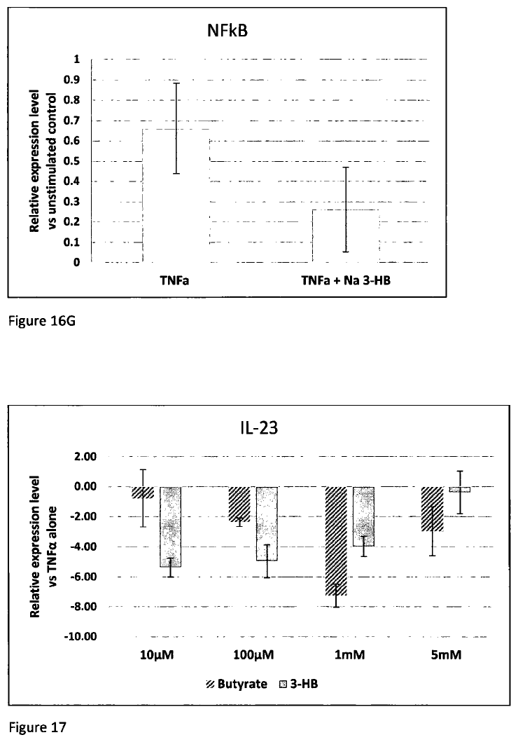

FIG. 17 shows the relative mRNA expression level of IL-23 in organoids treated with 60 ng/mL TNF-α and increasing concentrations of butyrate or (R)-3-HB. These data indicate that 10-100 μM concentrations of (R)-3HB may be effective at reducing expression of IL-23 in human gut mucosa. Il-23 is a key mediator of inflammation in IBD and is the target for several pharmaceutical monoclonal antibody drugs. This concentration range of (R)-3-Hb is achievable in the gut lumen using bacterial delivery as evidenced by in vitro gut modelling. (See FIG. 12). (R)-3-HB has a greater reducing impact on IL-23 expression at these concentrations than Butyrate.

### Example 6

### Evaluation of Survival in Stomach and Small Intestine Conditions

Spores of CHN-1 were resuspended in 1 mL PBS and inoculated into 9 mL of Gastric simulation medium (GSM) containing in g/L: arabinogalactan, 1, pectin, 2, xylan, 1, starch, 3, glucose, 0.4, yeast extract, 3, peptone, 1, mucin, 4, cysteine, 0.5, and pepsin, 1. The medium was adjusted to pH3 before autoclaving using 1M HCl. The GSM cultures were incubated anaerobically at 37° C. with 100 rpm agitation for 2 hours, before addition of 5 mL of pre-reduced pancreatic bile fluid containing in g/L: pancreatin, 3, dehydrated bile extract, 8, sodium bicarbonate, 10. Cultures were incubated anaerobically at 37° C. with 50 rpm agitation for 4 hours. The GSM was not prereduced but stored at 37° C. before transfer to the cabinet to mimick oxic conditions encountered in the stomach and reduction of oxygen encountered by travel through the gastrointestinal tract by susequent anaerobic incubation. Samples were taken at t=0, 2, 4, and 6 hours of incubation. Samples were serially diluted and spotted in 3 discreet 20 μL spots onto RCM agar plates. Plates were incubated anaerobically at 37° C. overnight before colony count to assess viability of spores in stomach and small intestine conditions.

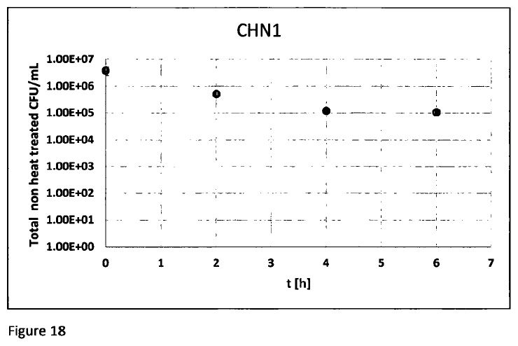

FIG. 18 shows that spores of CHN-1 survive stomach acid conditions and are then viable.

### Example 7

### Making Spores

C. butyricum CHN-1 spores are produced in nitrogen flushed agitated fermentation vessels of 2-100 L scale. The strain is handled in nitrogen and carbon dioxide flushed anaerobic workstations at 37° C. before inoculation into the fermentation vessel. CHN-1 is grown from frozen stocks (−80° C., modified Reinforced clostridial (RC) broth supplemented with 10% DMSO) on RC medium agar plates with visible colonies typically appearing after 12-16 hours of incubation. A single colony is used to inoculate modified RC broth. Serial dilutions 10⁻¹-10⁻⁸ of this cell suspension are prepared in modified RC broth and incubated for 12-16 hours. The highest diluted culture (typically 10⁻⁶) is used to inoculate fresh modified RC broth at a ratio of 1:10. This culture is grown to early exponential phase (typically 1½ hours) and transferred into serum bottles closed with rubber septa and metal caps to maintain anaerobicity. The culture is then inoculated into nitrogen flushed fermentation vessels (pH 6.5, 125 rpm agitation, 37° C.) by injection through the septum at a ratio of 1:10. After 24 hours, agitation is increased to 600 rpm for 15 minutes to remove biofilm formed on vessel parts. The cell/spore suspension is then harvested and stored at 4° C. until further processing. To purify spores, cell/spore suspension is repeatedly washed in ice-cold sterile water until >95% purity is confirmed by microscopy. Several washes (typically 10-15) of 20 min at 4° C. using a swing bucket centrifuge at 5,500 rpm and table top centrifuges at 10,000 rpm are carried out. Purified spores are aliquoted into individual doses and freeze-dried. Freeze-dried spores are stored in ventilated cabinets to prevent reintroduction of moisture.

### Example 8

### In Vivo and Pharmacokinetic Profiling of C. Butyricum

This study was conducted by EVOTEC International GmbH for CHAIN Biotechnology Ltd. After oral administration of C. butyricum CHN-1 to mice, bacterial levels were quantified by culture of spores and total bacteria on selective Butyricum isolation medium (BIM). Further, PCR was carried out to detect C. butyricum specific 16s-23s ISR and CHN-1 specific chromosomal insertion of codon optimised phaB (data not shown). Butyrate levels (data not shown) and levels of (R)-3-HB were assessed in colon tissue, faeces and plasma.

Methods:

Regulatory

All animal studies were performed under UK Home Office Licensure P2BC7D240 and with local ethical committee clearance. All studies were performed by technical staff who have completed parts A, B and C of the UK Home Office Personal License course and hold current personal licenses. All experiments were performed in dedicated Biohazard 2 facilities (this site holds a Certificate of Designation).

Animal Strain and Housing

Male mice used in these studies were supplied by Charles River UK and were specific pathogen free. The strain of mouse used was Hsd:ICR (CD-10), which is a well characterized outbred strain. Mice were 20-25 g on receipt at Evotec's facility and were allowed to acclimatize for minimum of 7 days prior to infection. Mice were approximately 30 g at the start of the study. Mice were housed in sterile individual ventilated cages exposing animals at all times to HEPA filtered sterile air. Mice had free access to food and water (sterile) and had sterile aspen chip bedding. The room temperature was 22° C.±1° C., with a relative humidity of 50-60% and maximum background noise of 56 dB. Mice were exposed to 12 hour light/dark cycles with dawn/dusk phases.

Preparation of Test Articles

a. Control Group

PBS was administered orally (PO) at a standard volume of 300 μL/mouse.

b. Test Article

C. butyricum CHN-1 spores were provided in freeze dried aliquots by the CHAIN Biotechnology Ltd. Each aliquot was reconstituted with 600 μL PBS and mice were given 300 μL PO. Upon reconstitution the test article formed a white viscous suspension.

Dosing and Sampling

Animals were dosed twice a day for five days. On days 0, 1, 2, 3, 4, 5 and 7 faecal samples were collected from three animals; 2-4 faecal pellets were collected from each animal per timepoint. On day 0 samples were additionally collected from three control group animals. Half the faecal samples were shipped to Cyprotex for analysis of compound levels whilst the remainder was quantitatively cultured to assess spore and total bacterial levels.

Terminal blood samples were then taken by cardiac puncture into potassium EDTA tubes. Blood samples were centrifuged as soon as possible at 13,000 rpm for 5 minutes to collect plasma. 0.5 mL plasma was added to the appropriate well of a 96 deep well plate, which was frozen at −20° C. between timepoints. Once the study was completed, the plate was shipped to Cyprotex for analysis of compound levels.

Upon euthanasia of the animal, the colon was dissected out and divided into two sections. One half was shipped to Cyprotex for analysis of compound levels whilst the remainder was quantitatively cultured to assess spore and total bacterial levels.

The study schedule is summarised in Table 2

TABLE 2
Study schedule (NA: not applicable)

| Day | 0 | 1 | 2 | 3 | 4 | 5 | 6 | 7 |
|---|---|---|---|---|---|---|---|---|
| Mice | 1-3 & | 4-6 | 7-9 | 10-12 | 13-15 | 16-18 | NA | 19-21 |
| PM | 22-24 | | | | | | | |

Spore dosing (3 = AM, 6 = AM & PM)

| | | | | | | | | |
|---|---|---|---|---|---|---|---|---|
| Control group | 3 | | | | | | | |
| (Mice 22-24) | | | | | | | | |
| Mice 1-3 | 3 | | | | | | | |
| Mice 4-6 | 6 | 3 | | | | | | |
| Mice 7-9 | 6 | 6 | 3 | | | | | |
| Mice 10-12 | 6 | 6 | 6 | 3 | | | | |
| Mice 13-15 | 6 | 6 | 6 | 6 | 3 | | | |
| Mice 16-18 | 6 | 6 | 6 | 6 | 6 | NA | | |
| Mice 19-21 | 6 | 6 | 6 | 6 | 6 | NA | NA | NA |

General Health Monitoring

The mice were monitored at a frequency appropriate for their clinical condition. Mouse weights were recorded to ensure animals remained within ethical limits.

Statistics

Data were analysed using StatsDirect v 2.7.8. A non-parametric Kruskal-Wallis test was used to test all pairwise comparisons (Conover-Inman).

In Vitro Analytical Method for Quantifying C. Butyricum in the Mouse Colon:

Growth Medium, Growth Conditions, and Quantification of Bacteria:

Prior to use, all growth media and diluents were pre-reduced at least overnight in an anaerobic workstation maintained at 37° C. and 65% relative humidity, and supplied with a gas mixture of 80/10/10 N2/H2/CO2. An aliquot of C. butyricum CHN-1 spores was suspended in peptone water and plated onto reinforced clostridial agar to confirm viability and serve as a validation control for the main study growth medium (see below) and for PCR assays (data not shown).

Following collection, colon samples were placed in the anaerobic workstation and transferred to 7 mL Precellys bead beater tubes containing 2 mL peptone water and 2.8 mm zirconium beads. Homogenisation took place outside the anaerobic chamber. Colon samples were homogenised in a Precellys Evolution bead beater for 10 s at 7,200 rpm, following which they were returned to the anaerobic chamber. Faecal pellets were transferred to 7 mL glass tubes containing 2 mL peptone water and four 3 mm glass beads, and samples were extensively vortex-mixed to homogeneity. Subsequent to homogenisation, samples were returned to the anaerobic chamber and split equally into aliquots for quantification of total bacteria (not heat treated) and spores (incubated at 65° C. for 30 min outside the anaerobic chamber). Samples were then serially diluted in peptone water prior to plating on Butyricum Isolation Medium (BIM) which comprised (per litre): NaCl, 0.9 g; CaCl2, 0.02 g; MgCl2.6H20, 0.02 g; MnCl2.4H2O, 0.01 g; CoCl2.6H2O, 0.001 g; KH2PO4, 7 g; K2HPO4, 7 g; iron sulfate, 0.001% (w/v); biotin, 0.0000005% (w/v); L-cysteine HCl, 0.5 g; glucose, 10 g; D-cycloserine, 250 mg; agar, 15 g. Agar plates were incubated for a minimum of 72 h prior to inspection and quantification of whitish, slightly raised colonies (approximately 1.5 to 3 mm in size) with undulated edges that were deemed to be C. butyricum.

Determination of Background Levels of 3-Hydroxybutyric Acid in CD-1 Mouse Colon, Faeces and Plasma by HPLC MS:

Injections were performed on a Sciex API 6600 TToF Hi-Res instrument employing uHPLC MS conditions that had been used previously at Cyprotex in the analysis of short-chain fatty acids. Under these conditions, it was possible to detect the presence of (R)-3-Hydroxybutyric acid by monitoring for the analyte accurate mass under negative ionization.

LC-MS Method

Mass Spec Conditions

Model: AB Sciex API 6600 TToF

Source/polarity: TIS/Neg

Curtain Gas (CUR): 40

Temperature (TEM): 700

Gas 1 (GS1): 80

Gas 2 (GS2): 50

IonSpray™ Voltage (IS): −4500

Detection: ToF MS

Mass range: 50-1000 Da

Accumulation time: 100 ms

Analyte: CY0000184922

Mass range (Da) 103.036-103.045

Declustering potential (DP) −80

Collison Energy (CE) −10

HPLC Conditions

System: Agilent HP1290 UPLC

Column: Phenomenex Luna Omega 1.6 μm 50×2.1

Mobile Phase A: 0.01% formic acid (aq)

Mobile Phase B: Methanol

Temperature: 50° C.

Analytical gradient

| Time | Flow rate | A | B |
|---|---|---|---|
| (min) | (μL/min) | (%) | (%) |
| 0.00 | 800 | 100 | 0 |
| 0.10 | 800 | 100 | 0 |
| 1.00 | 800 | 5 | 95 |
| 1.50 | 800 | 5 | 95 |
| 1.55 | 800 | 100 | 0 |
| 1.80 | 800 | 100 | 0 |

Injection Volume: 5 μL

Weak wash: 10% MeOH (aq)

Strong wash: 4:3:3 MeOH:IPA:Acetone+1% Acetic acid

Analysis of Plasma and Colon Samples

In brief, plasma samples (20 μL) were processed by adding 3 equivalents of acetonitrile (60 μL) to precipitate the plasma proteins. Samples were mixed and centrifuged at 3000 rpm for 30 minutes. The supernatant was then diluted in an excess of water to give overall dilutions of 40, 200 and 400-fold.

Tissue samples were weighed and 3 times the weight in water added. Samples were then homogenised using a Precellys homogeniser.

Samples were then precipitated with acetonitrile as detailed for the plasma samples (20 μL of sample and 60 μL of acetonitrile). The supernatant was diluted 10-fold with water resulting in an overall dilution factor of 160 for colon samples.

Processed samples were analysed in batches with calibration samples and quality control samples prepared in water.

Results

Clinical Observations

No tolerability issues were noted post treatment. No deterioration in clinical condition was noted in any animal and all animals gained weight throughout the study.

Colon and Faecal Burden

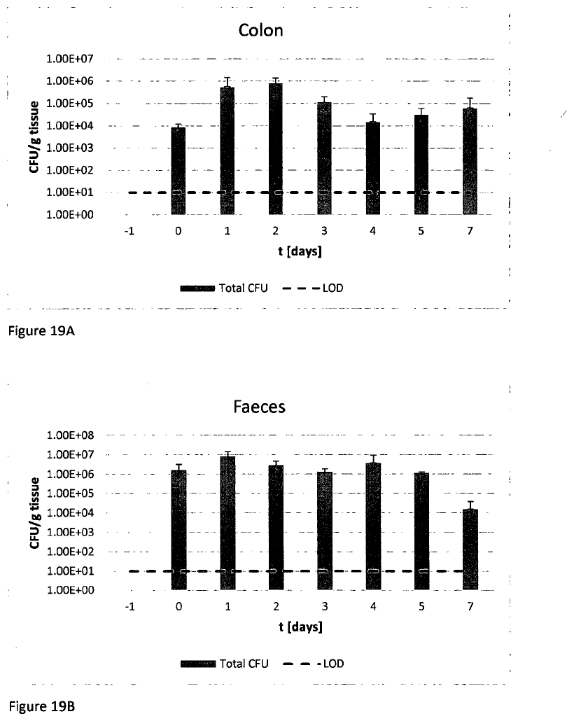

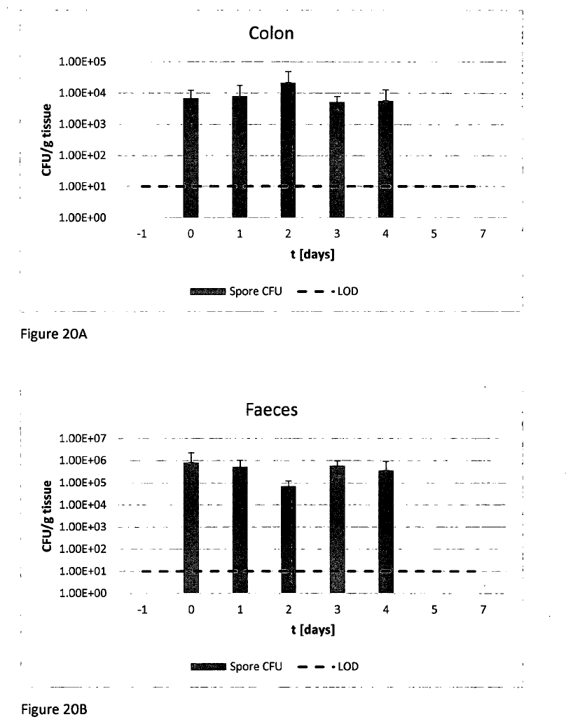

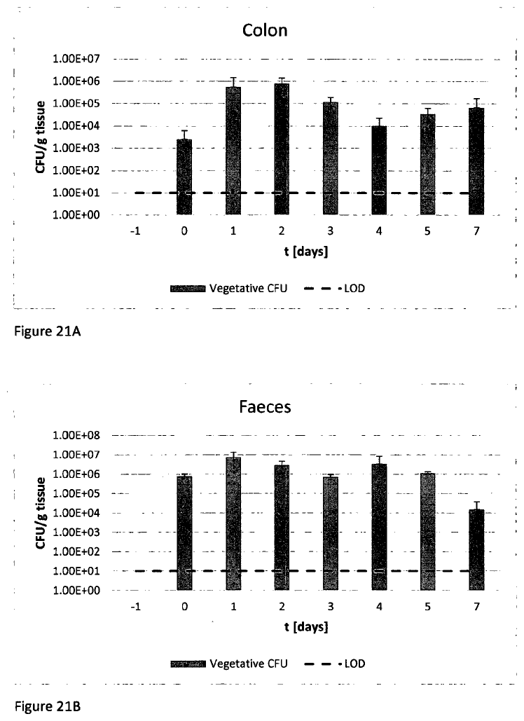

In the colon, bacteria were detected in samples from all animals dosed with C. butyricum, but not in the control group. Levels of bacteria peaked at 4.69 Log 10 CFU/mL/5.78 Log 10 CFU/g colon tissue at Day 2 post dosing. FIG. 19 shows colon and faecal total bacterial burden (CFU/g tissue) following administration of C. butyricum CHN-1. The geometric mean burden of each treatment is indicated by the horizontal bar. Note that for statistical and graphical purposes samples below the limit of detection were assigned an arbitrary value of 1. Limit of detection (LOD) is indicated by the labelled line. FIG. 20 shows colon and faecal spore burden following administration of C. butyricum CHN-1 (CFU/g tissue). The geometric mean burden of each treatment is shown. Error bars indicate the standard error. Note that for statistical and graphical purposes samples below the limit of detection were assigned an arbitrary value of 1. Limit of detection (LOD) is indicated by the labelled line. FIG. 21 shows the calculated viable colon and faecal vegetative CFU as determined by subtracting heat treated CFU from total CFU.

Summary of Bioanalytical Results

C. butyricum CHN-1 spores and total bacterial burden were quantitatively assessed in faecal samples and colon tissue after oral administration of spores. In the colon and faeces, spores were detected in samples from days 0, 1, 2 , 3 and 4 but no burden was detected at days 5 and 7 (after dosing had finished) or in the control group. Spores levels in samples from days 0, 1, 2, 3 and 4 were statistically significantly higher than levels detected in the control group. In the colon and faeces, bacteria were detected in samples from all animals dosed with C. butyricum CHN-1, but not in the control group.

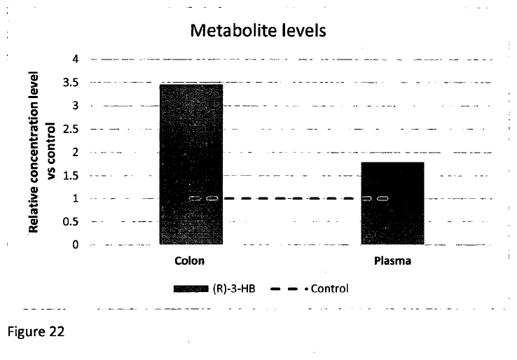

Butyrate (data not shown) and (R)-3-hydroxybutyrate were determined in colon samples as well as mouse plasma throughout the study. FIG. 22 shows relative concentration levels of (R)-3-hydroxybutyrate in samples assayed at day 5. In animals treated with C. butyricum CHN-1, levels of (R)-3-hydroxybutyrate in colon increased 2.46× (P=0.003) and in plasma levels increased from about 200 μM to about 300 μM (0.78×; P=0.01) in comparison to non-treated animal controls, i.e., an increase of about 100 μM.

### Example 9

### Improving Strain Yield

CHN-1 produces 0.2 g/L (R)-3-hydroxybutyrate by introduction of a single gene for acetoacetyl-CoA reductase. The production of (R)-3-hydroxybutyrate also requires the activity of two native enzymes, phosphotransbutyrylase (ptb) and butyrate kinase (buk). Buk has been reported to be the rate limiting step in production of short chain fatty acid butyrate (Appl Microbiol Biotechnol (2000) 53:545-552). In order to improve the titre of (R)-3-hydroxybutyrate in CHN-1, heterologous ptbl and bukl derived from C. saccheroperbutylacetonicum were introduced into CHN-1 on a pMTL82151 vector. Addition of heterologous genes for Ptb and Buk increased the titre of (R)-3-hydroxybutyrate from 0.2 g/L to 1.6 g/L. This presents a significant increase in product titre.

Allele coupled exchange procedures are carried out in order to generate stable integration of the heterologous genes into the CHN-1 genome.

### Example 10

### Improving Spore Manufacture

Spore manufacture of CHN-1 as described above had a yield of 10⁸ spores/mL of fermentation broth. In order to improve the spore yield and reduce required fermentation volumes, several experiments were carried out. No sporulation medium is described for Clostridia. However, by synchronising growth and subsequent spore formation, yield can be improved. To synchronise growth, cultures are grown directly from pure spore stocks instead of cell/spore mixtures as they occur on agar plates. Synchronised cultures are then used to inoculate fermentation vessels at the same ratios as described above. Base, but not acid is used to control pH at 6.5. Once mature spores are formed, cell lysis occurs, which increases the pH in the fermentation vessel. At this point, spores can be harvested from the vessel as described above. Using synchronised cultures increases the spore yield more than 10×.

1. A pharmaceutical composition for use in a method of treating a metabolic, neurodegenerative, neurological, inflammatory or autoimmune disease, disorder or condition or cancer in a subject, wherein the composition comprises a 3-hydroxybutyric acid (3-HB) delivery means, 3-HB or a combination thereof and the method comprises delivering the 3-HB delivery means, the 3-HB or combination thereof to the lower gastrointestinal (GI) tract.

2. A pharmaceutical composition for use as claimed in claim 1, wherein at least approximately 90% of the 3-HB is the (fl)-isomer ((R)-3-HB).

3. A pharmaceutical composition for use as claimed in claim 1, wherein the 3-HB delivery means is a biological delivery system or prodrug that delivers the 3-HB.

4. A pharmaceutical composition for use as claimed in claim 3, wherein the biological delivery system consists of anaerobic bacteria that produce 3-HB.

5. A pharmaceutical composition for use as claimed in claim 4, wherein the anaerobic bacteria are genetically engineered.

6. A pharmaceutical composition for use as claimed in claim 5, wherein the anaerobic bacteria are butyrate producing bacteria comprising a non-native gene capable of expressing (R)-3-hydroxybutyryl-CoA dehydrogenase.

7. A pharmaceutical composition for use as claimed in claim 4, wherein the anaerobic bacteria are spore forming obligate anaerobes.

8. A pharmaceutical composition for use as claimed in claim 4, wherein the bacteria are Clostridia bacteria.

9. A pharmaceutical composition for use as claimed in claim 4, wherein the bacteria are from cluster I, IV and/or XIVa of Clostridia.

10. A pharmaceutical composition for use as claimed in claim 4, wherein the bacteria are from the Clostridium genus.

11. A pharmaceutical composition for use as claimed in claim 4, wherein the bacteria are Clostridium butyricum.

12. A pharmaceutical composition for use as claimed in claim 4, wherein the bacteria have native genes encoding phosphotransbutyrylase and/or butyrate kinase.

13. A pharmaceutical composition for use as claimed in claim 4, wherein the bacteria produce (R)-3-HB as the sole fermentation product.

14. A pharmaceutical composition for use as claimed in claim 4, wherein the bacteria produce (R)-3-HB in combination with acetate, lactate, and/or butyrate as fermentation products.

15. A pharmaceutical composition for use as claimed in claim 1, wherein the method comprises delivering the 3-HB delivery means, the 3-HB or combination thereof to anaerobic sections of the lower GI tract, preferably to the colon and/or terminal ileum.

16. A pharmaceutical composition for use as claimed in claim 1, wherein the pharmaceutical composition is formulated for modified-release.

17. A pharmaceutical composition for use as claimed in claim 1, wherein the pharmaceutical composition is administered orally.

18. A pharmaceutical composition for use as claimed in claim 16, wherein the pharmaceutical composition comprises a modified-release layer or coating surrounding a core comprising the 3-HB delivery means, the 3-HB or combination thereof.

19. A pharmaceutical composition for use as claimed in claim 15, wherein the pharmaceutical composition is formulated to deliver the 3-HB delivery means, the 3-HB or combination thereof to the lower GI tract at a pH of between about 5 and about 7.

20. A pharmaceutical composition for use as claimed in claim 15, wherein the pharmaceutical composition is formulated to deliver the 3-HB delivery means, the 3-HB or combination thereof to the lower GI tract between 5 and 40 hours after oral administration with food.

21. A pharmaceutical composition for use as claimed in claim 1, wherein the subject is a human.

22. A pharmaceutical composition for use as claimed in claim 1, wherein the subject is fasting and/or is on a ketogenic diet.

23. A method of treating a metabolic, neurodegenerative, neurological, inflammatory or autoimmune disease, disorder or condition or cancer in a subject comprising administering to the subject a pharmaceutical composition comprising a 3-hydroxybutyric acid (3-HB) delivery means, 3-HB or a combination thereof wherein the 3-HB delivery means, the 3-HB or combination thereof is delivered to the lower gastrointestinal (GI) tract.
